SEQUENCE LISTING (71 sequences)

SEQ ID NO: 1 - DNA, 2435 bp, Homo sapiens
  misc_feature: artificial sequence, Translation start at 209; stop at 2435
gagattttatcgggagcagtgaggtgactttggcagctaacaggccactagtatcctactaaagcttttgtctggataggagcaacatgcatgtttacagtcttgcagtgtgctgagagctggtggccagtgggactgagtgagctgtgtgccgtgtattgacccgcttcctagtcctgaattcctttcagaagctccggcagggaggatgatacagtcagacaaaggagcagatccaccagacaagaaggacatgaagctttctacagccaccaatccacagaatggcctctcccagatcctgaggcttgtgctgcaagagctgagtctgttctacagcagagatgtgaatggagtgtgtctcttgtacgatctcctccactcgccgtggcttcaggctctgctaaagatttatgactgcctccaggaatttaaagaaaagaaactagttcctgccacaccacatgcacaggtgttatcctatgaggtagtggagttattacgtgaaacccctacttcccctgagatccaagagctgagacaaatgctccaggctccacacttcaaggccttgctcagtgcccatgacacgatagctcagaaagattttgaaccccttctccctccactgccagacaatatccctgagagtgaggaagcaatgaggattgtttgtttagtgaaaaaccaacagcccctgggagccaccatcaagcgccacgagatgacaggggacatcttggtggccaggatcatccacggtgggctggcggagagaagtgggttgctatatgctggagacaaactggtagaagtgaatggagtttcagttgagggactggaccctgaacaagtgatccatattctggccatgtctcgaggcacaatcatgttcaaggtggttccagtctctgaccctcctgtgaatagccagcagatggtgtacgtccgtgccatgactgagtactggccccaggaggatcccgacatcccctgcatggacgctggattgcctttccagaagggggacatcctccagattgtggaccagaatgatgccctctggtggcaggcccgaaaaatctcagaccctgctacctgcgctgggcttgtcccttctaaccaccttctgaagaggaagcaacgggaattctggtggtctcagccgtaccagcctcacacctgcctcaagtcaaccctatcaatttctatggaagaagaagatgacatgaagattgatgagaaatgtgtggaagcagatgaagaaacatttgaatctgaggaactttcagaagacaaggaggagtttgttggctacggtcagaagttctttatagctggcttccgccgcagcatgcgcctttgtcgcaggaagtctcacctcagcccgctgcatgccagtgtgtgctgcaccggcagctgctacagtgcagtgggtgccccttacgaggaggtggtgaggtaccagcgacgcccttcagacaagtaccgcctcatagtgctcatgggaccctctggtgttggagtaaatgagctcagaagacaacttattgaatttaatcccagccattttcaaagtgctgtgccacacactactcgtactaaaaagagttacgaaatgaatgggcgtgagtatcactatgtgtccaaggaaacatttgaaaacctcatatatagtcacaggatgctggagtatggtgagtacaaaggccacctgtatggcactagtgtggatgctgttcaaacagtccttgtcgaaggaaagatctgtgtcatggacctagagcctcaggatattcaaggggttcgaacccatgaactgaagccctatgtcatatttataaagccatcgaatatgaggtgtatgaaacaatctcggaaaaatgccaaggttattactgactactatgtggacatgaagttcaaggatgaagacctacaagagatggaaaatttagcccaaagaatggaaactcagtttggccaattttttgatcatgtgattgtgaatgacagcttgcacgatgcatgtgcccagttgttgtctgccatacagaaggctcaggaggagcctcagtgggtaccagcaacatggatttcctcagatactgagtctcaatgagacttcttgtttaatgctggagttttaacactgtacccttgatacagcgatccatagttgcaatctaaaacaacagtatttgacccattttaatgtgtacaactttaaaagtgcagcaatttattaattaatcttatttgaaaaaaatttttattgtatggttatgtggttacctattttaacttaattttttttcctttacctcatatgcagctgtggtagaaatatgaataatgttaagtcactgagtatgagaacctttcgcagatttcacatgatctttttaagatttaaataaagagctttcctaaat

SEQ ID NO: 2 - DNA, 320 bp, Homo sapiens
  misc_feature: genomic DNA, Exon from 1 to 108
gagattttatcgggagcagtgaggtgactttggcagctaacaggccactagtatcctactaaagcttttgtctggataggagcaacatgcatgtttacagtcttgcaggtaagagaccttggcaaataatcctcagttaccagaagatgtatccataactgcctagcttgcctgtcagtttttaatagctaaagatataaatctgggtaatctaactcaaatggcttagtttcattttaactcaaatgatatggggaattttatgatcttgaaagagcaggttttgcttcgagaagccatttcttcagtatggaataatg

SEQ ID NO: 3 - DNA, 512 bp, Homo sapiens
  misc_feature: genomic DNA, Exon from 173 to 352
aaacatggagttagggggagcattttatgcaatagtcgttttctctttcacgccactggtgatggttaagagtaggcaccacaggggaagactgtgtttcatttgatgtgtatcccagtgtgtagcacagggcctggcttgctgaggaaatgctattgaaaatatattccagtgtgctgagagctggtggccagtgggactgagtgagctgtgtgccgtgtattgacccgcttcctagtcctgaattcctttcagaagctccggcagggaggatgatacagtcagacaaaggagcagatccaccagacaagaaggacatgaagctttctacagccaccaatccacagaatggtatgtgtcaccaggactccttttctagaccagaaagtaatatcacctctgacatgtgatcaaatgaataggcagaaatcctgacagacttactgtgatccctatgaggatcttgtacatttttggttgcactactgccctaccagtgataactttaagaa

SEQ ID NO: 4 - DNA, 448 bp, Homo sapiens
  misc_feature: genomic DNA, Exon from 165 to 286
acaaagtaagaggtggaacagggcttgaagtcagatcttttggcctgagatccagtgtcatttccactcctggtgagaccccatggcatgccccagctatctgagttgcctttcacatttacacccgcacctgccaccccatctctgctctcttcctttcctaggcctctcccagatcctgaggcttgtgctgcaagagctgagtctgttctacagcagagatgtgaatggagtgtgtctcttgtacgatctcctccactcgccgtggcttcaggctctgctaaaggtgagtgcttctttgctcggaagcctttgcttgctgaaggggttgtggggagtgtgtagaaaatgacagcttcagtccattcaggctggatagtggaatagtttataaacaacagaaattgatatctcacagttctgtaggccaggaagtccaaaatccagt

SEQ ID NO: 5 - DNA, 448 bp, Homo sapiens
  misc_feature: genomic DNA, Exon from 206 to 283
taagcttttgaagcatcggggccaccaaactcaagttcatttctctttggcaactagagacacaacttactaaacaccaaccacaccgtgctgtgcagccattggtgcagttgcctggggtgtttcttctctttgagagtcttaaatccaaaatggcaatagtcatattatcaatatcaattctccctcccttgtccttctgcagatttatgactgcctccaggaatttaaagaaaagaaactagttcctgccacaccacatgcacaggtgttatcctatgaggtaaggagattttattccacaggatagtagagctctgatgtggtgccattttccccacattgctagttcaaatgaattaaaggttctaaggaaaagttttattgatgactatgcatctaataaatgtttctaattgaactttaatataaggaagaacattggctg

SEQ ID NO: 6 - DNA, 384 bp, Homo sapiens
  misc_feature: genomic DNA, Exon from 165 to 245
ggtaggctttaatggatggctttatagatgaaaaagaaggctccagtaatagctttttaaaggtcaatatcatgttagtatgtatgttatccagcctgggtgaggttaagtaggtgataaagattttttaaaatttttataatgtatccttttccatgaaccaggtagtggagttattacgtgaaacccctacttcccctgagatccaagagctgagacaaatgctccaggctccacacttcaaggcaagtgcctgctaaaatagaaaagatgtccccatctggcacatagacaaagttgggaaggagaaatatatgtgatggaaaatgttctctctgaatagatgttctattactgtacacggttactgaccaacagattgta

SEQ ID NO: 7 - DNA, 448 bp, Homo sapiens
  misc_feature: genomic DNA, Exon from 133 to 264
cgcactgtgtctggcatgtctgtattggtgtttgttgttgttgctgtgtcttagatagtattgagttactatcttctagaggggtttggcccatgtgtgacatttgctcaccttttccttccctgtgcccaggccttgctcagtgcccatgacacgatagctcagaaagattttgaaccccttctccctccactgccagacaatatccctgagagtgaggaagcaatgaggattgtttgtttagtgaaaaaccaacagcccctggtaaggaaatcattttttatctttccatttagggtaagcttaggttaattgtgaaccaaattatatctagtggttacttgggcagtagccttgcctgcgatcacatatacagtgataataacggctgtcaactctgcaagttttgcctgtggtttcaaacatattacatgtcacggtgttttct

SEQ ID NO: 8 - DNA, 448 bp, Homo sapiens
  misc_feature: genomic DNA, Exon from 166 to 247
cattgattgaaagaccagagctgcattgattgaaagaccagagctgcattgattgaaagaccagagctgcattgattgaaagaccagagctgcattgattgagggaagccacctggaaaatggtcatgtcaggtaacagagggatctcgtctattctctcttcagggagccaccatcaagcgccacgagatgacaggggacatcttggtggccaggatcatccacggtgggctggcggagagaagtggtaagctggagcagctgggattgagagttaccagaaaaacaggaaacccttgactgtttaggcttctttctagagaaatcccttttttttctttttttttttctttttttttttttgagatggagtcttgctctgtcgcccaggctggagtgcagtggcgtgatctcggctcactgcaagctccacctctggggtttgcca

SEQ ID NO: 9 - DNA, 448 bp, Homo sapiens
  misc_feature: genomic DNA, Exon from 162 to 247
atgtaagttggaataaccagctttcttttctattattattttatattaaacatttttagagcatgcttgggttagtgagttaaatagctatcgaggtagctactgctatttttatcctacttctttgtatctttctttgttttttgttactgtctgcctagggttgctatatgctggagacaaactggtagaagtgaatggagtttcagttgagggactggaccctgaacaagtgatccatattctggtaaatcttctttttgcctttttgttaatgacttggagaaatgccaaggctgaactgggaccatcaagcccacgtgtgtgcactgggatgtaccggggactcaagttctcttggcagctttctccctccaggctcccagaccttgtctgtcacccatgtcacttgctgacctccctcctctaccccgagaagttctggtcc

SEQ ID NO: 10 - DNA, 384 bp, Homo sapiens
  misc_feature: genomic DNA, Exon from 158 to 229
ccatttctggatggtgacagctgcagagcccttgtgaaaggctcttgggggattttaccatgagacctggatacattgcactgtaactctgtccaccgagccccagtaaccctgctagctccatgattgtcatcctttctcctctcttattttccaggccatgtctcgaggcacaatcatgttcaaggtggttccagtctctgaccctcctgtgaatagccagcagatggtaagaatttactgagccttcaatctcacacacagtaaatccccaagtaacagcaactaaatatgatgcgtaataatcctatcctttgtactgtgttggacctggattcaagactgtgttggatatttttcaatactgatggcccgagaagcaaa

SEQ ID NO: 11 - DNA, 448 bp, Homo sapiens
  misc_feature: genomic DNA, Exon from 138 to 334
gggtggagaggaaagataggagtagcaggtggaggagtgggagaaatggttttaagtcatgatggcccatgggcaagggttcttcggatggcaccattaggcaccttctgatagcgtcattatgcacctgccatcaggtgtacgtccgtgccatgactgagtactggccccaggaggatcccgacatcccctgcatggacgctggattgcctttccagaagggggacatcctccagattgtggaccagaatgatgccctctggtggcaggcccgaaaaatctcagaccctgctacctgcgctgggcttgtcccttctaaccaccttctgaagaggtaaggaacgtcaccactcctggactcagggctgaaccatcaggaaacaaaatgtttttcttgggtttctgttacctcaagatgagataaagagggacaagcagatgaatgaac

SEQ ID NO: 12 - DNA, 320 bp, Homo sapiens
  misc_feature: genomic DNA, Exon from 152 to 216
atttggagaagcaatcacccttttcacttgagtgaaggcagcagaattctaagaaacattctgtttgtcgttgctctgggtctgtttcatctaggttaacaaagagtggtttttgtttgttttttgtcgcatggttttttcccccccataggaagcaacgggaattctggtggtctcagccgtaccagcctcacacctgcctcaagtcaaccctatgtgagtattgcaactgcccgacaggttcttcctgtttgcaataaagaccatggcattgcagtaaataaagagtctaattgatgtgaggctggccatgccacatg

SEQ ID NO: 13 - DNA, 320 bp, Homo sapiens
  misc_feature: genomic DNA, Exon from 161 to 178
cttactaaatcttccctgaatttctagagaataaacccagaatactaattacaataatttttgcacattacatttcttattgtaaattaatctgagaaaatatagtacagatactgtgttctttttatcccccctgcttcaatcatttgcttgtactcagcaatttctatggaagaaggtaagaaatagtatttaggaaaaaactcttatctccaaagtcttttagaaatttcttgtagtttaaagaattcactttaattcagttcagctatttattaagctcttcctatatacctagtagtgtgatagtcattattaag

SEQ ID NO: 14 - DNA, 384 bp, Homo sapiens
  misc_feature: genomic DNA, Exon from 179 to 217
catggtttcaccatgtcggtcaagttggtctcgaactcttgatctcaggtgatccgcccgcctcggcctcccagagtgctgggatcacaggcatgagccaccatgcctggccgggaattttctttttaatgcagacacattttaaattctgtttctccctttctatactcttttatagaagatgacatgaagattgatgagaaatgtgtggaagcaggtaacattttctcttgattgctttgctgttagaagaaatatgaagcatgtcaattatagattatctgaagcagaggtgtccaaaggggccatgggcctttcctctagaaatgtgtaaaatgaccctccacccccatctatcttctgtagttctggcacttggaagga

SEQ ID NO: 15 - DNA, 320 bp, Homo sapiens
  misc_feature: genomic DNA, Exon from 110 to 130
gtggtgtatgggcagaagtaggggccagagaattagacttaaaatatagactcagtgtagatggtcatgtaataacatttttgatttttgcctccatgaaaaatcatagatgaagaaacatttgaatctggtaagtaaaaaatgagtatttggtactgatttttaaatgtatattctaaattttgatgcaatttatacacatatttataataactgtttaaatatatcaacattaaaaaattaaaaagtaactgcgtgtatcccacatcatgttgtcaacctcaaatatacattataaaatttatttttaattttaattt

SEQ ID NO: 16 - DNA, 320 bp, Homo sapiens
  misc_feature: genomic DNA, Exon from 174 to 188
cagtcccaatcatgtggtgatcatttgccttgccaggccttacccgagttaccttttgctagtggtgacgtgcacgtcttgcttatgtcatttgccttgatttgatggctaacatgatcttcttaaaggcttaactttttcatgtctgtttctgcacttacccaaatatccagaggaactttcagaaggtaattgtttttatttcctagatataccaaatagaactatgtttaagatctttcagtgcctcaaaaatgaatacttgactggataatgtttaagatgaagatacggaatttgttgttgtttatggttttccc

SEQ ID NO: 17 - DNA, 320 bp, Homo sapiens
  misc_feature: genomic DNA, Exon from 170 to 211
ataggatgaaaaatgcttagaacattcggtgagccactcaaggataaattcaactctgctgccgtctactaaggtggtcacttgaaaagttgaaaatgatttcatgaatttattctgaataaacttctcgctctcacatattctgctccatctgttctttgtgtttcagacaaggaggagtttgttggctacggtcagaagttctttataggtaggtgataaattaacaagaggtgggtctctgtcacttgttaaattatgtttccaaacctgacactgttttgaaagtttcttttgctaatgaacatttccagacctgt

SEQ ID NO: 18 - DNA, 512 bp, Homo sapiens
cccaagacaatgcctggcccagagcaggtgctagatgggctagcacagggggcattttcatatttttccctcatattacttcccatcttctaacttcagacagacctgactatattaatgaacactttaggatcatggttgctacatatttcatcaggtgtgaagctacaagtgatctctcctgcctggttcttacgttctgtgcacttcccctccctagctggcttccgccgcagcatgcgcctttgtcgcaggaagtctcacctcagcccgctgcatgccagtgtgtgctgcaccggcagctgctacagtgcagtgggtgccccttacgaggaggtggtgaggtaccagcgacgcccttcagacaagtaccgcctcatagtgctcatgggtatgtcccagcatgcactgtctctcctcctccttgagaagtcttccttctagattcaggtgtcttgcattgggaataatggtgaaagtagaactctttatggacccccatacaaatacct

SEQ ID NO: 19 - DNA, 384 bp, Homo sapiens
  misc_feature: genomic DNA, Exon from 160 to 240
ttctggggttcttccaatttatgagaaaggaagttacataatttccctaaaaatatttgctctcaagtttcttcagtagaaggactaaaatgataattccatcacataattatatttatccacatctgatgatttctgtgtgtgactttttgtgtttaggaccctctggtgttggagtaaatgagctcagaagacaacttattgaatttaatcccagccattttcaaagtgctgtgccacgtatgtttagttctgctttcataatggtttgtgttttggtaaaactttctttgctgatctcatttaactatgtcattccatctttgttgtaaaagtatacaacaccagggatagttcttaagtatttctaaccatatttatttt

SEQ ID NO: 20 - DNA, 448 bp, Homo sapiens
  misc_feature: genomic DNA, Exon from 200 to 293
tcagtaaaggtttatagactaactgattttgatacgagaacttatcaccaattcaggcttcttctttttagttctagcattttatctccttgattatatattcatttatttattttgattagatatctttattcaaatgcatattggtaatcaaagaattctgaagacactgaaacctttcattccctttttctgatagacactactcgtactaaaaagagttacgaaatgaatgggcgtgagtatcactatgtgtccaaggaaacatttgaaaacctcatatatagtcacaggtaaagtagaggttcagaagctgattcttacctcttgttgttttacatttgaaatagattccctatttttatgtattttccaaatctcctgggtaattccttttgtttctgaggagttaagcaagaaatgtacatcgatatacagcacaccaact

SEQ ID NO: 21 - DNA, 448 bp, Homo sapiens
  misc_feature: genomic DNA, Exon from 133 to 241
atctattcattctttctgtgttaataaagtccacatatttatattcaactctagtgcagtttatcctcatgttactactaataatattttccttgtagaaagtgttctgttttgtttggcctgctcttgcaggatgctggagtatggtgagtacaaaggccacctgtatggcactagtgtggatgctgttcaaacagtccttgtcgaaggaaagatctgtgtcatggacctagagcctcaggtgggtccatggtggaatatttatgtccccaaacaatgaatgcgtatcatccattttttgtgcacatgctgtaggttatagttgagacatttattctgttagccttttaagaataaggccatttcccatatataagatcttacttaacgtgtcaattgacaacattttacttttagttgggaaagaagtcttgcttctcagacagaa

SEQ ID NO: 22 - DNA, 448 bp, Homo sapiens
  misc_feature: genomic DNA, Exon from 164 to 298
agctacttgggaggctgagatgggtggatcgtttgagcctgggaagctgaggctacagtgaactgtgattgcaccacagcactccagcctgggtgacagagcaagaccatgtctcaaaacaaaacaaacaaaaaataaatgtgcatttaaattttctgtgtaggatattcaaggggttcgaacccatgaactgaagccctatgtcatatttataaagccatcgaatatgaggtgtatgaaacaatctcggaaaaatgccaaggttattactgactactatgtggacatgaagttcaaggtaagagcaagtcaaaaactactgtattgctttcagtggcttctgcgtgggagagatctgggttgggctgggccaaggatctctgatctcattgtcctcctcctcctttttgaccccctctccaaaaggccctcaataaaatggtttact

SEQ ID NO: 23 - DNA, 704 bp, Homo sapiens
  misc_feature: genomic DNA, Exon from 197 to 704
ttttctagtttgctggttttgtagaattttgaaaaaatatttttgaaactttattgaaaatcatctgtgcaaaattttcggaccttactgtttttatacatagtttcacaactgaatgtgacagcataacaaactgtattttttccatttgtccaattaagtctgtactatccatatttttctatttctcctaaaggatgaagacctacaagagatggaaaatttagcccaaagaatggaaactcagtttggccaattttttgatcatgtgattgtgaatgacagcttgcacgatgcatgtgcccagttgttgtctgccatacagaaggctcaggaggagcctcagtgggtaccagcaacatggatttcctcagatactgagtctcaatgagacttcttgtttaatgctggagttttaacactgtacccttgatacagcgatccatagttgcaatctaaaacaacagtatttgacccattttaatgtgtacaactttaaaagtgcagcaatttattaattaatcttatttgaaaaaaatttttattgtatggttatgtggttacctattttaacttaattttttttcctttacctcatatgcagctgtggtagaaatatgaataatgttaagtcactgagtatgagaacctttcgcagatttcacatgatctttttaagatttaaataaagagctttcctaaat

SEQ ID NO: 24 - protein, 637 aa, Homo sapiens
MIQSDKGADPPDKKDMKLSTATNPQNGLSQILRLVLQELSLFYSRDVNGVCLLYDLLHSPWLQALLKIYDCLQEFKEKKLVPATPHAQVLSYEVVELLRETPTSPEIQELRQMLQAPHFKALLSAHDTIAQKDFEPLLPPLPDNIPESEEAMRIVCLVKNQQPLGATIKRHEMTGDILVARIIHGGLAERSGLLYAGDKLVEVNGVSVEGLDPEQVIHILAMSRGTIMFKVVPVSDPPVNSQQMVYVRAMTEYWPQEDPDIPCMDAGLPFQKGDILQIVDQNDALWWQARKISDPATCAGLVPSNHLLKRKQREFWWSQPYQPHTCLKSTLSISMEEEDDMKIDEKCVEADEETFESEELSEDKEEFVGYGQKFFIAGFRRSMRLCRRKSHLSPLHASVCCTGSCYSAVGAPYEEVVRYQRRPSDKYRLIVLMGPSGVGVNELRRQLIEFNPSHFQSAVPHTTRTKKSYEMNGREYHYVSKETFENLIYSHRMLEYGEYKGHLYGTSVDAVQTVLVEGKICVMDLEPQDIQGVRTHELKPYVIFIKPSNMRCMKQSRKNAKVITDYYVDMKFKDEDLQEMENLAQRMETQFGQFFDHVIVNDSLHDACAQLLSAIQKAQEEPQWVPATWISSDTESQ

SEQ ID NO: 25 - DNA, 1190 bp, Homo sapiens
  misc_feature: artificial sequence, Translation start at 48, stop at 638
ataaacattgggctgcacatagagacttaattttagatttagacaaaatggaaattatttcatcaaaactattcattttattgactttagccacttcaagcttgttaacatcaaacattttttgtgcagatgaattagtgatstccaatcttcacagcaaagaaaattatgacaaatattctgagcctagaggatacccaaaaggggaaagaagcctcaattttgaggaattaaaagattggggaccaaaaaatgttattaagatgagtacacctgcagtcaataaaatgccacactccttcgccaacttgccattgagatttgggaggaacgttcaagaagaaagaagtgctggagcaacagccaacctgcctctgagatctggaagaaatatggaggtgagcctcgtgagacgtgttcctaacctgccccaaaggtttgggagaacaacaacagccaaaagtgtctgcaggatgctgagtgatttgtgtcaaggatccatgcattcaccatgtgccaatgacttattttactccatgacctgccagcaccaagaaatccagaatcccgatcaaaaacagtcaaggagactgctattcaagaaaatagatgatgcagaattgaaacaagaaaaataagaaacctggagcctgtccctaaagctgtggcctgtaatctacaaatggctctatagcgaagaccacacggaagagtagctacatacacttcatcagctatggatcatcaacggcaatttttccttgtcagtacagctataatagtatcttgaaagttgtaaaaaaattaaagcatatttgttacgtaaagttaaaatgatttttgtctgaataaaaaaaaagcattgcaaatgctttagaaatctctgataatggagagagagacagaggaccctcctcactaccctatataaaaatcattggcacagttacacttaataaaaaaaattaaacagaagagcaccctgaaaaacattatgatggaaattaaatagtatgccagaataacatggttgacaaataagtgaacaaggattaaaaatcacttacaaacgtgtttctgtacaccctttctatcgtgtcaaatgttaatgaatctgtgatcaattgaaatgtaaatgtctgtgtaaaactacaaaataaaaactcttagactttagggagaaaagaaaa

SEQ ID NO: 26 - DNA, 256 bp, Homo sapiens
  misc_feature: genomic DNA, Exon from 1 to 185
ataaacattgggctgcacatagagacttaattttagatttagacaaaatggaaattatttcatcaaaactattcattttattgactttagccacttcaagcttgttaacatcaaacattttttgtgcagatgaattagtgatstccaatcttcacagcaaagaaaattatgacaaatattctgaggtaagttttttaaatctctctaatgtgagtagcattaattacataatattaatcctaagtctaatgatttt

SEQ ID NO: 27 - DNA, 512 bp, Homo sapiens
  misc_feature: genomic DNA, Exon from 62 to 462
gggtttaaatctgttgcttataacaacagtatgttattgtaatggtcatttctaattatagcctagaggatacccaaaaggggaaagaagcctcaattttgaggaattaaaagattggggaccaaaaaatgttattaagatgagtacacctgcagtcaataaaatgccacactccttcgccaacttgccattgagatttgggaggaacgttcaagaagaaagaagtgctggagcaacagccaacctgcctctgagatctggaagaaatatggaggtgagcctcgtgagacgtgttcctaacctgccccaaaggtttgggagaacaacaacagccaaaagtgtctgcaggatgctgagtgatttgtgtcaaggatccatgcattcaccatgtgccaatgacttattttactccatgacctgccagcaccaagaaatccagaatcccgatcaaaaacagtcaaggtaaatacctggaaaccagtcaaagtgcatgggcagttatatagaggtgg

SEQ ID NO: 28 - DNA, 768 bp, Homo sapiens
  misc_feature: genomic DNA, Exon from 115 to 718
acacaattcaactcaagtataattaggcagttaggactatggcttgtatttgtatacacacttgcatgctgttgttctgatgggtgacaacattttatactgcttacattttaggagactgctattcaagaaaatagatgatgcagaattgaaacaagaaaaataagaaacctggagcctgtccctaaagctgtggcctgtaatctacaaatggctctatagcgaagaccacacggaagagtagctacatacacttcatcagctatggatcatcaacggcaatttttccttgtcagtacagctataatagtatcttgaaagttgtaaaaaaattaaagcatatttgttacgtaaagttaaaatgatttttgtctgaataaaaaaaaagcattgcaaatgctttagaaatctctgataatggagagagagacagaggaccctcctcactaccctatataaaaatcattggcacagttacacttaataaaaaaaattaaacagaagagcaccctgaaaaacattatgatggaaattaaatagtatgccagaataacatggttgacaaataagtgaacaaggattaaaaatcacttacaaacgtgtttctgtacaccctttctatcgtgtcaaatgttaatgaatctgtgatcaattgaaatgtaaatgtctgtgtaaaactacaaaataaaaactcttagactttagggagaaaagaaaaaggcaactatgagttacctcttttagtgtctcctctatctacatccagaa

SEQ ID NO: 29 - protein, 196 aa, Homo sapiens [sequence omitted]

SEQ ID NO: 30 - DNA, 1188 bp, Homo sapiens
  misc_feature: artificial sequence, Translation start at 347, stop at 604
acacacaacggggtttcggggctgtggaccctgtgccaggaaaggaagggcgcagctcctgcaatgcggagcagccagggcagtgggcaccaggctttagcctccctttctcaccctacagagggcaggcccttcagctccattctcctccaaggctgcagagggggcaggaattgggggtgacaggagagctgtaaggtctccagtgggtcattctgggcccagagatgggtgctgaagctcccacgcctgcctgtgaaaatggagtcctctctcacctgggagagccaggtgctgccccgagaaggatgcatttatggcttcrtgaagtctttcctgacccccgatgctgctgactatagagacaaagtctcactatgttgctcaggctggtcttgaactcctggcctcaagcgatcctcccacctyagcctcccaaagwgttgggattatagacatgagccactgcacctggccgaccttgggcaagttcttaaacccttcaaagcctcatttttctccaatcayaaaagggaaagatggtaatattttccccwccaaattcttgtcggatgccctcacagaattgagattatgtacgtaaaacaccaggtgcctaacccggcacagagcaggagggctaagcgtgacatccagcacgtggtcagtggaatccagtattcctacccacctctctagtctcccctccacccctctccctttcagaggcaccaagctgcttgtggtcttgtctattcccactccctgcctgactgaacattttctccacctcctgatcatcagcagcagaaactggctgctcttcctcctgggtagacagccagactgtatttcccagctgcccctgcagtgagatgtggccatcggagccagcattggccaatggactctgcatgggagtgacgcatgcwgcctccaggcttgtccctaaaacctcccacgtgtcctcsgcctgctcttcccacytccaaggagcacggcaattgtggaagacccagattagtgatggcagaaccatagatgggaggaacctgggtccctgacttaaagtatcatggatttggatgttcccttagtgagaaataaacttccattgtgtttaagcctttatttgtttatagttggttacagcaactgccttcttttaattaaaacactcctgctgct

SEQ ID NO: 31 - protein, 85 aa, Homo sapiens
MLLTIETKSHYVAQAGLELLASSDPPTSASQSVGIIDMSHCTWPTLGKFLNPSKPHFSPITKGKDGNIFPTKFLSDALTELRLCT

SEQ ID NO: 32 - DNA, 560 bp, Homo sapiens
  misc_feature: genomic DNA, Exon from 101 to 460
tatatgggaatgagccagctgcaccgctgctgacagtggctgggataatcctccctgagctgttccaaggattagtcctgctgccctgtgcccagctcccacacaacggggtttcggggctgtggaccctgtgccaggaaaggaagggcgcagctcctgcaatgcggagcagccagggcagtgggcaccaggctttagcctccctttctcaccctacagagggcaggcccttcagctccattctcctccaaggctgcagagggggcaggaattgggggtgacaggagagctgtaaggtctccagtgggtcattctgggcccagagatgggtgctgaagctcccacgcctgcctgtgaaaatggagtcctctctcacctgggagagccaggtgctgccccgagaaggatgcatttatggcttcatgaagtctttcctgacccccgatgctgctgactataggtaagtctgagcaaatctgggggagcctcatcttggcatgagaaagagatggcttcttctaagcccactggccgtgatcccaggattataacacattctg

SEQ ID NO: 33 - DNA, 405 bp, Homo sapiens
  misc_feature: genomic DNA, Exon from 101 to 305
catgagaggtagtataatatagaggatatgtgtgcttactaagaggctgcctgtctgaccttggacaagttctttttatttatttatttattttttatagagacaaagtctcactatgttgctcaggctggtcttgaactcctggcctcaagcgatcctcccaccttagcctcccaaagagttgggattatagacatgagccactgcacctggccgaccttgggcaagttcttaaacccttcaaagcctcatttttctccaatcataaaagggaaagatggtaatattttcccctccaaattcttgtaagtattaaacattgtatatgtattttgaacacgattaagctctaaacacttgttaggaagcaggagtagcatttgaaacaaacagctcttttcccac

SEQ ID NO: 34 - DNA, 821 bp, Homo sapiens
  misc_feature: genomic DNA, Exon from 101 to 721
aagtattaaacattgtatatgtattttgaacacgattaagctctaaacacttgttaggaagcaggagtagcatttgaaacaaacagctcttttcccacaggtcggatgccctcacagaattgagattatgtacgtaaaacaccaggtgcctaacccggcacagagcaggagggctaagcgtgacatccagcacgtggtcagtggaatccagtattcctacccacctctctagtctcccctccacccctctccctttcagaggcaccaagctgcttgtggtcttgtctattcccactccctgcctgactgaacattttctccacctcctgatcatcagcagcagaaactggctgctcttcctcctgggtagacagccagactgtatttcccagctgcccctgcagtgagatgtggccatcggagccagcattggccaatggactctgcatgggagtgacgcatgctgcctccaggcttgtccctaaaacctcccacgtgtcctccgcctgctcttcccacttccaaggagcacggcaattgtggaagacccagattagtgatggcagaaccatagatgggaggaacctgggtccctgacttaaagtatcatggatttggatgttcccttagtgagaaataaacttccattgtgtttaagcctttatttgtttatagttggttacagcaactgccttcttttaattaaaacactcctgctgcttcatgttgctggaatgcttgtaaccctgccctgcttcaccagggtaactcctacttggcctttaagtttatctctgctgtcacaccgtccagaaagcctt

SEQ ID NO: 35 - DNA, 1514 bp, Homo sapiens
  misc_feature: artificial sequence, Translation start at 155, stop at 1192
gaaagtccagccatctgttacctgcgttgcttcctggggrgggatagtccacctggaggcattcggagacccagtgattgtgctccgyggagcctgggctgtgccccgcgttgactgcctcatagataccctacgaaccccaaatgccagctgcatgagaaaagggactcaccttctggttccctgcctggaagaggaagagctggcattgcacaggagacggctggacatgtctgaggcactgccctgcccgggcaaggagacccccaccccaggctgcaggctgggggccctgtattgggcctgtgtccacaatgatcccacccagctccaagccatactggatggtggggtctccccagaggaggccacccaggtggacagcaatgggaggacaggcctcatggtcgcatgctaccacggcttccagagtgttgtggccctgctcagccactgtcctttccttgatgtgaaccagcaggacaaaggaggggacacggccctcatgttggctgcccaagcaggccacgtgcctctagtgagtctcctgctcaactactatgtgggcctggacctggaacgccgggaccagcgggggctcacggcgttaatgaaggctgccatgcggaaccgctgtgctgacctgacagcagtggaccctgttcggggcaagacggccctggaatgggcagtgctgaccgacagcttcgacaccgtgtggaggattcggcagctgctgaggcggccccaagtggagcagcttagccagcactacaagcccgagtggccggccttgtccgggctcgtggcccaggcccaggcccaggcccaggttgccccttcactcctagaacggctgcaggctaccttgagcctcccctttgccccgtctcctcaggaggggggtgttctggaccaccttgtgactgccacaaccagcctggccagtcccttcgtcaccactgcctgccacactctgtgccctgaccatccaccttcgctgggcacccgaagcaagtccgtgccagagctgttagtgccagccgaagcccagtccttcaggacaccaaagtctggcccttcctctctggcgataccaggagctcaggatagagaagaggaaacaggaggaggaggccagaatggcacagaagtaggggaagatgggataggacaggctgggaacaggtaatcaggcccctcccagggcttctttcccctctggagtgcctccggcctccccatccacctctgcctaagtaaatctgctctcaacctatatatatacaaggtcattcattctagcattgtttgcaagagtgaaagagtggaaacacccgaagtgtccatcagtaagggacaggctagattgattacggatgtaattgctgtccatccatacagagcatactctacagtgtattctaaaataagactaaggaagctgtttatattctgatatgaaactaccatcaagatgtataaagtaaaaataactaaggagtggaacagtg

SEQ ID NO: 36 - DNA, 1544 bp, Homo sapiens
  misc_feature: artificial sequence, Translation start at 155, stop at 1222
gaaagtccagccatctgttacctgcgttgcttcctggggrgggatagtccacctggaggcattcggagacccagtgattgtgctccgyggagcctgggctgtgccccgcgttgactgcctcatagataccctacgaaccccaaatgccagctgcatgagaaaagggactcaccttctggttccctgcctggaagaggaagagctggcattgcacaggagacggctggacatgtctgaggcactgccctgcccgggcaaggagacccccaccccaggctgcaggctgggggccctgtattgggcctgtgtccacaatgatcccacccagctccaagccatactggatggtggggtctccccagaggaggccacccaggtggacagcaatgggaggacaggcctcatggtcgcatgctaccacggcttccagagtgttgtggccctgctcagccactgtcctttccttgatgtgaaccagcaggacaaaggaggggacacggccctcatgttggctgcccaagcaggccacgtgcctctagtgagtctcctgctcaactactatgtgggcctggacctggaacgccgggaccagcgggggctcacggcgttaatgaaggctgccatgcggaaccgctgtgagtgcgtggccaccctcctcatggcaggtgctgacctgacagcagtggaccctgttcggggcaagacggccctggaatgggcagtgctgaccgacagcttcgacaccgtgtggaggattcggcagctgctgaggcggccccaagtggagcagcttagccagcactacaagcccgagtggccggccttgtccgggctcgtggcccaggcccaggcccaggcccaggttgccccttcactcctagaacggctgcaggctaccttgagcctcccctttgccccgtctcctcaggaggggggtgttctggaccaccttgtgactgccacaaccagcctggccagtcccttcgtcaccactgcctgccacactctgtgccctgaccatccaccttcgctgggcacccgaagcaagtccgtgccagagctgttagtgccagccgaagcccagtccttcaggacaccaaagtctggcccttcctctctggcgataccaggagctcaggatagagaagaggaaacaggaggaggaggccagaatggcacagaagtaggggaagatgggataggacaggctgggaacaggtaatcaggcccctcccagggcttctttcccctctggagtgcctccggcctccccatccacctctgcctaagtaaatctgctctcaacctatatatatacaaggtcattcattctagcattgtttgcaagagtgaaagagtggaaacacccgaagtgtccatcagtaagggacaggctagattgattacggatgtaattgctgtccatccatacagagcatactctacagtgtattctaaaataagactaaggaagctgtttatattctgatatgaaactaccatcaagatgtataaagtaaaaataactaaggagtggaacagtg

SEQ ID NO: 37 - protein, 345 aa, Homo sapiens
MRKGTHLLVPCLEEEELALHRRRLDMSEALPCPGKETPTPGCRLGALYWACVHNDPTQLQAILDGGVSPEEATQVDSNGRTGLMVACYHGFQSVVALLSHCPFLDVNQQDKGGDTALMLAAQAGHVPLVSLLLNYYVGLDLERRDQRGLTALMKAAMRNRCADLTAVDPVRGKTALEWAVLTDSFDTVWRIRQLLRRPQVEQLSQHYKPEWPALSGLVAQAQAQAQVAPSLLERLQATLSLPFAPSPQEGGVLDHLVTATTSLASPFVTTACHTLCPDHPPSLGTRSKSVPELLVPAEAQSFRTPKSGPSSLAIPGAQDREEETGGGGQNGTEVGEDGIGQAGNR

SEQ ID NO: 38 - protein, 355 aa, Homo sapiens
MRKGTHLLVPCLEEEELALHRRRLDMSEALPCPGKETPTPGCRLGALYWACVHNDPTQLQAILDGGVSPEEATQVDSNGRTGLMVACYHGFQSVVALLSHCPFLDVNQQDKGGDTALMLAAQAGHVPLVSLLLNYYVGLDLERRDQRGLTALMKAAMRNRCECVATLLMAGADLTAVDPVRGKTALEWAVLTDSFDTVWRIRQLLRRPQVEQLSQHYKPEWPALSGLVAQAQAQAQVAPSLLERLQATLSLPFAPSPQEGGVLDHLVTATTSLASPFVTTACHTLCPDHPPSLGTRSKSVPELLVPAEAQSFRTPKSGPSSLAIPGAQDREEETGGGGQNGTEVGEDGIGQAGNR

SEQ ID NO: 39 - DNA, 183 bp, Homo sapiens
  misc_feature: genomic DNA, Exon from 1 to 143
gaaagtccagccatctgttacctgcgttgcttcctggggrgggatagtccacctggaggcattcggagacccagtgattgtgctccgyggagcctgggctgtgccccgcgttgactgcctcatagataccctacgaaccccaagtaagaaaaaacgacgaccctctctccgtgagtctcactg

SEQ ID NO: 40 - DNA, 462 bp, Homo sapiens
  misc_feature: genomic DNA, Exon from 108 to 358
gggataaatgttttccctggggcaagggctgtgcacttcgcagctgctgggtcccctccctaggatccagggagacactcactactcctctccattctgtgttttagatgccagctgcatgagaaaagggactcaccttctggttccctgcctggaagaggaagagctggcattgcacaggagacggctggacatgtctgaggcactgccctgcccgggcaaggagacccccaccccaggctgcaggctgggggccctgtattgggcctgtgtccacaatgatcccacccagctccaagccatactggatggtggggtctccccagaggaggccacccaggtggacagcaatgggagggtgagatgtcctggcttcccagaacagctgggggcatctttgcatccccaccacaccgtcctggcctggctccctgagaggggttcaggggcaatacctcctgc

SEQ ID NO: 41 - DNA, 308 bp, Homo sapiens
  misc_feature: genomic DNA, Exon from 89 to 218
ctctgggacagatatgggtttagagggtgcaaggggccctggagtggcccagggggaaagcaggggatctgagctgcccctccctcagacaggcctcatggtcgcatgctaccacggcttccagagtgttgtggccctgctcagccactgtcctttccttgatgtgaaccagcaggacaaaggaggggacacggccctcatgttggctgcccaagcaggtgtgaggctgctgcaccccacttccgacagcccccttttgatgcagacagggcctcagccccacccttgttgcacggtgttctacacca

SEQ ID NO: 42 - DNA, 231 bp, Homo sapiens
  misc_feature: genomic DNA, Exon from 49 to 159
tcatcaccccctttcctggggaccaagcttacccttgctgccctgcaggccacgtgcctctagtgagtctcctgctcaactactatgtgggcctggacctggaacgccgggaccagcgggggctcacggcgttaatgaaggctgccatgcggaaccgctgtgagtgcgtggccaccctcctcatggcaggtgtgcggggcctggaccggggtgtgtggcctccagtccctc

SEQ ID NO: 43 - DNA, 231 bp, Homo sapiens; identical to SEQ ID NO: 42
  misc_feature: genomic DNA, Exon from 49 to 189

SEQ ID NO: 44 - DNA, 588 bp, Homo sapiens
  misc_feature: genomic DNA, Exon from 98 to 499
aatgtaacccacatcagtcttgctcctaaagaatctgcccttccacaaatcaccaacccctatcccgccccatgtcaccccctgtgctccttcccaggtgctgacctgacagcagtggaccctgttcggggcaagacggccctggaatgggcagtgctgaccgacagcttcgacaccgtgtggaggattcggcagctgctgaggcggccccaagtggagcagcttagccagcactacaagcccgagtggccggccttgtccgggctcgtggcccaggcccaggcccaggcccaggttgccccttcactcctagaacggctgcaggctaccttgagcctcccctttgccccgtctcctcaggaggggggtgttctggaccaccttgtgactgccacaaccagcctggccagtcccttcgtcaccactgcctgccacactctgtgccctgaccatccaccttcgctgggcacccgaagcaagtccgtgccagagctgttaggtactgccccgccccctcccctggttccccagtccccgccagggagtccccagaggtccccgtgggtcttcgtcccctaccagagccct

SEQ ID NO: 45 - DNA, 503 bp, Homo sapiens
  misc_feature: genomic DNA, Exon from 27 to 503
ccaaggcatcctcatcctcccaccagtgccagccgaagcccagtccttcaggacaccaaagtctggcccttcctctctggcgataccaggagctcaggatagagaagaggaaacaggaggaggaggccagaatggcacagaagtaggggaagatgggataggacaggctgggaacaggtaatcaggcccctcccagggcttctttcccctctggagtgcctccggcctccccatccacctctgcctaagtaaatctgctctcaacctatatatatacaaggtcattcattctagcattgtttgcaagagtgaaagagtggaaacacccgaagtgtccatcagtaagggacaggctagattgattacggatgtaattgctgtccatccatacagagcatactctacagtgtattctaaaataagactaaggaagctgtttatattctgatatgaaactaccatcaagatgtataaagtaaaaataactaaggagtggaacagtg

SEQ ID NO: 46 - DNA, 18 bp, Artificial Sequence
  primer
ctcacatccttctcagcc

SEQ ID NO: 47 - DNA, 19 bp, Artificial Sequence
  primer
gtggaatgtcagggaaatc

SEQ ID NO: 48 - DNA, 18 bp, Artificial Sequence
  primer
tgactgcctccaggaatt

SEQ ID NO: 49 - DNA, 18 bp, Artificial Sequence
  primer
ttacgaaatgaatgggcg

SEQ ID NO: 50 - DNA, 18 bp, Artificial Sequence
  primer
aggctctaggtccatgac

SEQ ID NO: 51 - DNA, 19 bp, Artificial Sequence
  primer
atgtgaaatctgcgaaagg

SEQ ID NO: 52 - DNA, 18 bp, Artificial Sequence
  primer
cgtgccatgactgagtac

SEQ ID NO: 53 - DNA, 18 bp, Artificial Sequence
  primer
aactgcagtgggtaccag

SEQ ID NO: 54 - DNA, 19 bp, Artificial Sequence
  primer
tctgagcctagaggatacc

SEQ ID NO: 55 - DNA, 18 bp, Artificial Sequence
  primer
gatctcagaggcaggttg

SEQ ID NO: 56 - DNA, 20 bp, Artificial Sequence
  primer
tgctgtgaagattggagatc

SEQ ID NO: 57 - DNA, 36 bp, Artificial Sequence
  primer
ggccacgcgtcgactagtacgggnngggnngggnng

SEQ ID NO: 58 - DNA, 20 bp, Artificial Sequence
  primer
ggccacgcgtcgactagtac

SEQ ID NO: 59 - DNA, 20 bp, Artificial Sequence
  primer
agcttgaagtggctaaagtc

SEQ ID NO: 60 - DNA, 20 bp, Artificial Sequence
  primer
tgatctccaatcttcacagc

SEQ ID NO: 61 - DNA, 18 bp, Artificial Sequence
  primer
tgtgccaggaaaggaagg

SEQ ID NO: 62 - DNA, 19 bp, Artificial Sequence
  primer
tagtcagcagcatcggggg

SEQ ID NO: 63 - DNA, 21 bp, Artificial Sequence
  primer
agcaagttcagcctggttaag

SEQ ID NO: 64 - DNA, 18 bp, Artificial Sequence
  primer
atgttcagtcaggcaggg

SEQ ID NO: 65 - DNA, 18 bp, Artificial Sequence
  primer
ttcttgtcggatgccctc

SEQ ID NO: 66 - DNA, 18 bp, Artificial Sequence
  primer
cggaaccgctgtgagtgc

SEQ ID NO: 67 - DNA, 18 bp, Artificial Sequence
  primer
taggcagaggtggatggg

SEQ ID NO: 68 - DNA, 18 bp, Artificial Sequence
  primer
ggccactcgggcttgtag

SEQ ID NO: 69 - DNA, 18 bp, Artificial Sequence
  primer
gtgcaatgccagctcttc

SEQ ID NO: 70 - DNA, 18 bp, Artificial Sequence
  primer
tgccaagctgttagtgcc

SEQ ID NO: 71 - DNA, 18 bp, Artificial Sequence
  primer
catgctaccacggcttcc

(19) United States
(12) Patent Application Publication
(10) Pub. No.: US 2003/0054446 A1
(43) Pub. Date: Mar. 20, 2003

(54) Novel retina-specific human proteins C7orf9, C12orf7, MPP4 and F379

(76) Inventors: Bernard H. F. Weber, Wuerzburg (DE); Heidi Stoehr, Wuerzburg (DE)

Correspondence Address: BURNS DOANE SWECKER & MATHIS L L P, POST OFFICE BOX 1404, ALEXANDRIA, VA, 22313-1404, US

(21) Appl. No.: 09/995,793

(22) Filed: Nov. 29, 2001

Related U.S. Application Data
(60) Provisional application No. 60/253,751, filed on Nov. 29, 2000.

(51) Int. Cl. C12Q 1/68; C07H 21/04; C12N 9/00; C12P 21/02; C12N 5/06

(52) U.S. Cl. 435/69.1; 435/183; 435/320.1; 435/325; 536/23.2; 435/6

(57) ABSTRACT

The present invention relates to the novel human retina-specific proteins called C7orf9, C12orf7, MPP4 and F379, and isolated nucleic acid molecules encoding said proteins. Also provided are vectors, host cells, antibodies and recombinant methods for producing these human proteins. The invention further relates to diagnostic and therapeutic methods useful for diagnosing and treating macular degeneration, e.g. AMD.

### FIELD OF THE INVENTION

The present invention relates to gene expression in human retinal tissue and particularly to the novel retina-specific proteins C7orf9, C12orf7, MPP4 and F379 associated with macular degeneration including age-related macular degeneration (AMD) and the genes encoding C7orf9, C12orf7, MPP4 and F379.

### BACKGROUND OF THE TECHNOLOGY

First described in 1855, age-related macular degeneration (AMD) is now recognized as the most common cause of visual morbidity in the developed world The prevalence of AMD in persons over 52 was found to be 9% increasing to more than 25% in persons over the age of 75. Projected estimates indicate that by the year 2020 as many as 7.5 million individuals over 65 years may suffer from central vision loss due to AMD. As the population of older people in industrialized countries increases, the associated social and economic consequences of AMD are destined to increase in the next millenium unless preventive or therapeutic treatments can be devised.

Histologically, an increasing accumulation of yellowish lipofuscin-like particles within the retinal pigment epithelium (RPE) can be observed with age. This likely represents an early stage in the evolution of AMD which is followed by secondary complications frequently associated with loss of visual acuity. It is thought that the lipofuscin-like deposits represent remnants of undigested phagocytosed photoreceptor outer segment membranes which, in the normal physiological processes, are excreted basally through Bruch's membrane into the choriocapillaris. Over time, incomplete digestion and accumulation of lipofuscin-like particles affect Bruch's membrane and lead to its progressive destruction as seen by electron microscopy as an abnormal thickening of the inner collagenous layer of the membrane. The deposits in the RPE and Bruch's membrane consist largely of lipids although their exact composition may vary between individuals with some deposits revealing more polar phospholipids while others contain predominantly apolar neutral lipids.

These individual differences in drusen composition are thought to be the basis for the clinical heterogeneity in AMD. While some patients present with an ingrowth of vessels from the choriocapillaris through Bruch's membrane, others show pigment epithelial detachment due to exudation underneath the RPE, and a third group of patients experiences a slow decrease of visual loss due to atrophic changes in the RPE and the overlying sensory neuroretina. Although much less common, the exudative/neovascular form of AMD accounts for more than 80% of blindness with a visual acuity of ≦20/200.

AMD is a complex disease caused by exogenous as well as endogenous factors. In addition to environmental factors, several personal risk factors such as hypermetropia, light skin and iris colour, elevated serum cholesterol levels, hypertension or cigarette smoking have been suggested. A genetic component for AMD has been documented by several groups and has lead to the hypothesis that the disease may be triggered by environmental/individual factors in those persons who are genetically predisposed. The number of genes which, when mutated, can confer susceptibility to AMD is so far not known. The photoreceptor-specific ATP-binding cassette (ABCR) gene may represent the first example of a gene predisposing to AMD, although methodological problems in study design and interpretation of data have given rise to controversy.

Extensive research is currently in progress and is directed towards the identification of genes conferring susceptibility to AMD. However, the late onset of symptoms generally in the 7th decade of life as well as the clinical and likely genetic heterogeneity makes it difficult to apply conventional approaches for the identification of the genes predisposing to AMD.

The above discussed limitations and failings of the prior art to provide retina-specific genes predisposing to macular degeneration like AMD, e.g. gene variants which correlate with the occurrence of macular degeneration or genes showing aberrant expression which is correlated with the occurrence of macular degeneration has created a need for genes (markers) which can be used diagnostically, prognostically and therapeutically over the course of this disease. The present invention fulfills such a need by the provision of C7orf9, C12orf7, MPP4 and F379 and the genes encoding C7orf9, C12orf7, MPP4 and F379: The genes encoding C7orf9, C12orf7, MPP4 and F379 are expressed in retinal tissue, but not in other tissues tested. The identification of said genes was achieved by the use of a new computer-assisted strategy which aimed at the genome-wide identification of genes that are expressed exclusively or predominantly in the human retina and made use of the in silico expression information enclosed in the expressed sequence tag (EST) clusters of the publicly available UniGene dataset (Schuler, Mol.Med. 75 (1997), 694-698). Genes uniquely or preferentially active in the retina should play an important finctional role in this highly differentiated tissue and therefore may causally be involved in the etiology of AMD and other retinal degenerative diseases.

### SUMMARY OF THE INVENTION

The present invention is based on the isolation of genes which might be causally involved in the etiology of AMD and other retinal degenerative diseases, C7orf9, C12orf7, MPP4 and F379. The cloning and sequencing of C7orf9, C12orf7, MPP4 and F379 should facilitate the analysis of their possible role in retinal disease and the development of methods for the diagnosis and prophylactic/therapeutic treatments of macular degeneration, e.g. AMD.

The present invention, thus, provides C7orf9, C12orf7, MPP4 and F379 proteins, respectively, as well as nucleic acid molecules encoding said proteins and, moreover, an antisense RNA, a ribozyme and an inhibitor, which allow to inhibit the expression or the activity of C7orf9, C12orf7, MPP4 and/or F379.

In one embodiment, the present invention provides a diagnostic method for detecting macular degeneration or a predisposition for said disease.

In another embodiment, the present invention provides a method of (prophylactically) treating macular degeneration.

Finally, the present invention provides a method of gene therapy comprising introducing into cells of a subject an expression vector comprising a nucleotide sequence encoding C7orf9, C12orf7, MPP4 and/or F379 or the above mentioned antisense RNA or ribozyme, in operable linkage with a promoter.

### FIGURES

FIG. 1 Expression analysis of MPP4. (A) Northern blot probed with an MPP4 specific probe originating from the 3′UTR. (B) RT-PCR analysis in human tissues with oligonucleotide primer pair A128aF/A128aR located in exon 19 and 20 of the MPP4 gene, respectively. The beta-glucuronidase gene served as a control to ensure RNA quality and equal loading.

FIG. 2 Expression of C7orf9. (A) Northern blot probed with a C7orf9 specific probe originating from the 5′ end of the gene. (B) RT-PCR analysis in human tissues with oligonucleotide primer pair A129F3/A129R located in exon 1 and 2 of the C7orf9 gene, respectively.

FIG. 3 Expression analysis of F379. (A) Northern blot probed with an F379 specific probe originating from the 3′ end of the gene. (B) RT-PCR analysis in human tissues with oligonucleotide primer pair A071F/A071R located in exon 1 of the F379 gene.

FIG. 4 Expression of C12orf7. RT-PCR analysis in human tissues with oligonucleotide primer pair A038F4/038R3 located in exon 3 and 5 of the C12orf7 gene.

FIG. 5 Seq. ID No.1. Shows the nucleotide sequence of the MPP4 cDNA.

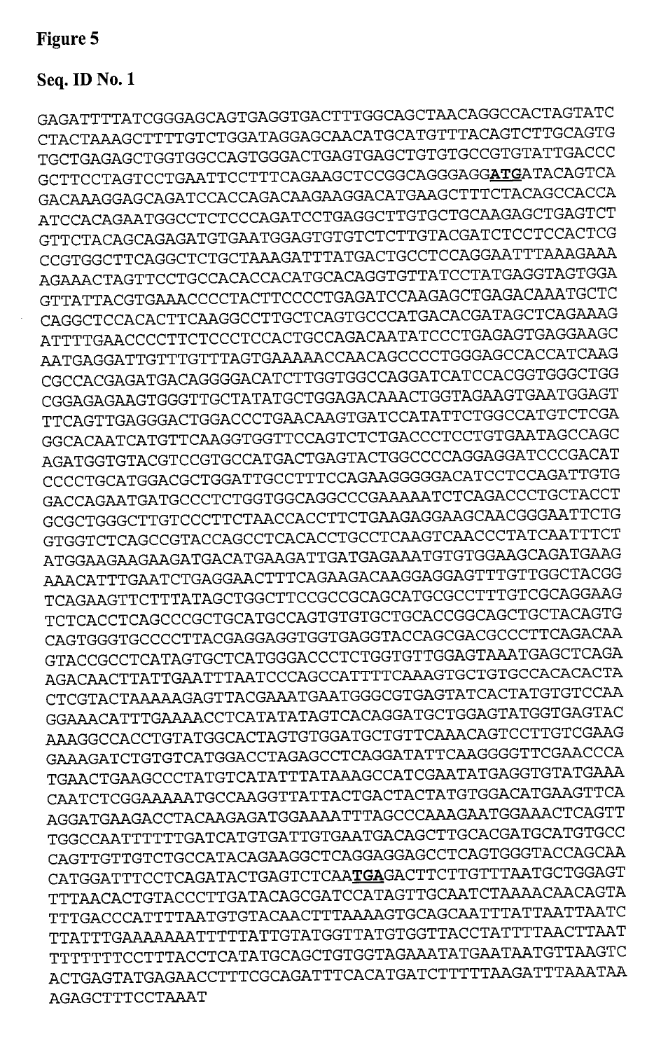

FIG. 6a Seq. ID Nos. 2-5. Shows the nucleotide sequence of the exon/intron organization of exons 1-4 of the MPP4 gene.

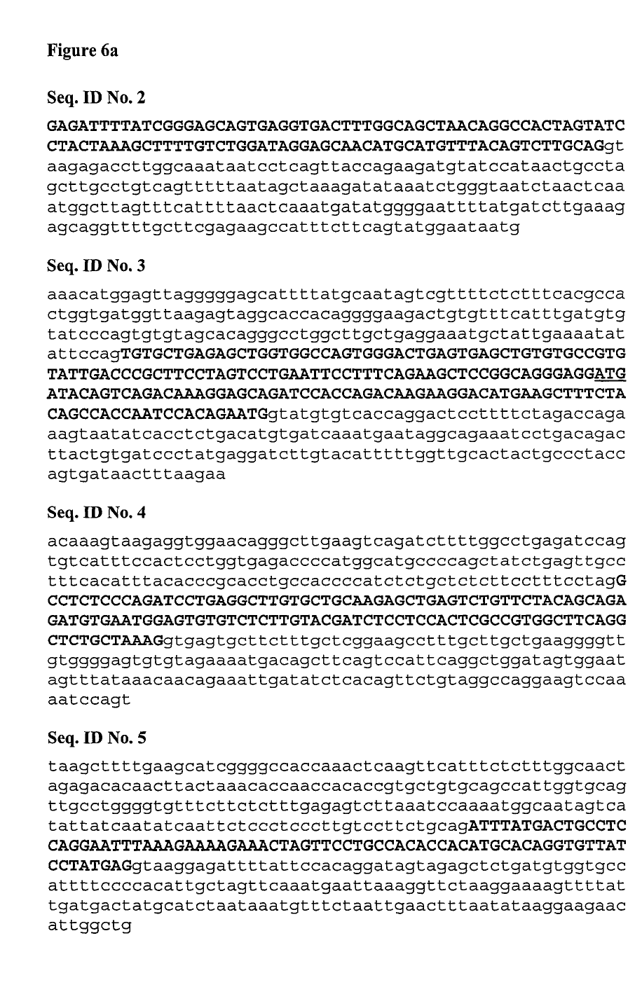

FIG. 6b Seq. ID Nos. 6-9. Shows the nucleotide sequence of the exon/intron organization of exons 5-8 of the MPP4 gene.

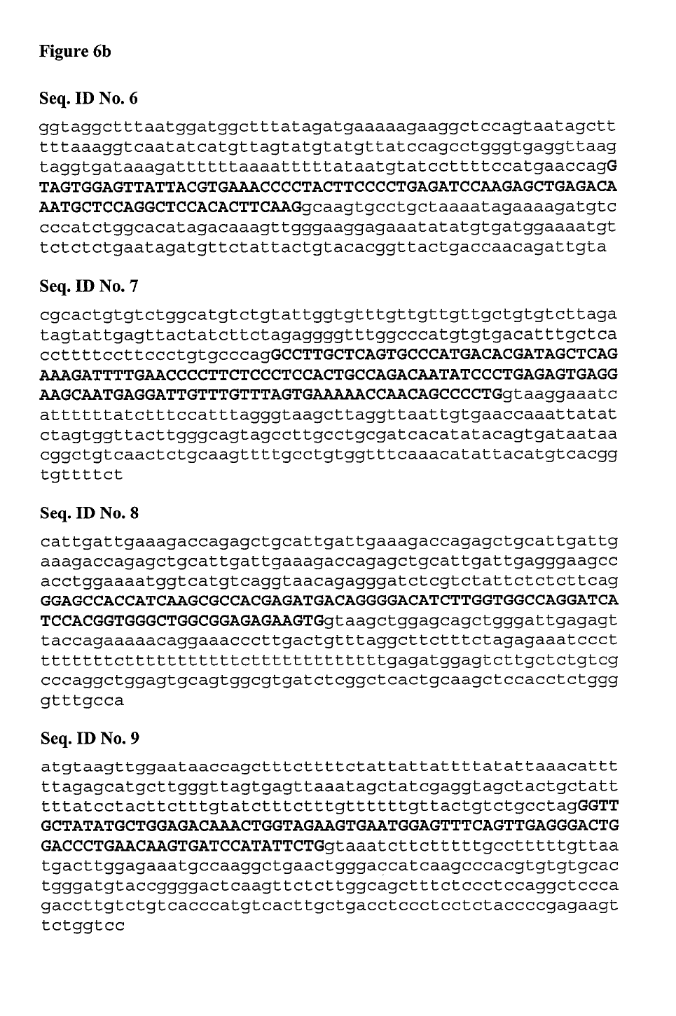

FIG. 6c Seq. ID Nos. 10-14. Shows the nucleotide sequence of the exon/intron organization of exons 9-13 of the MPP4 gene.

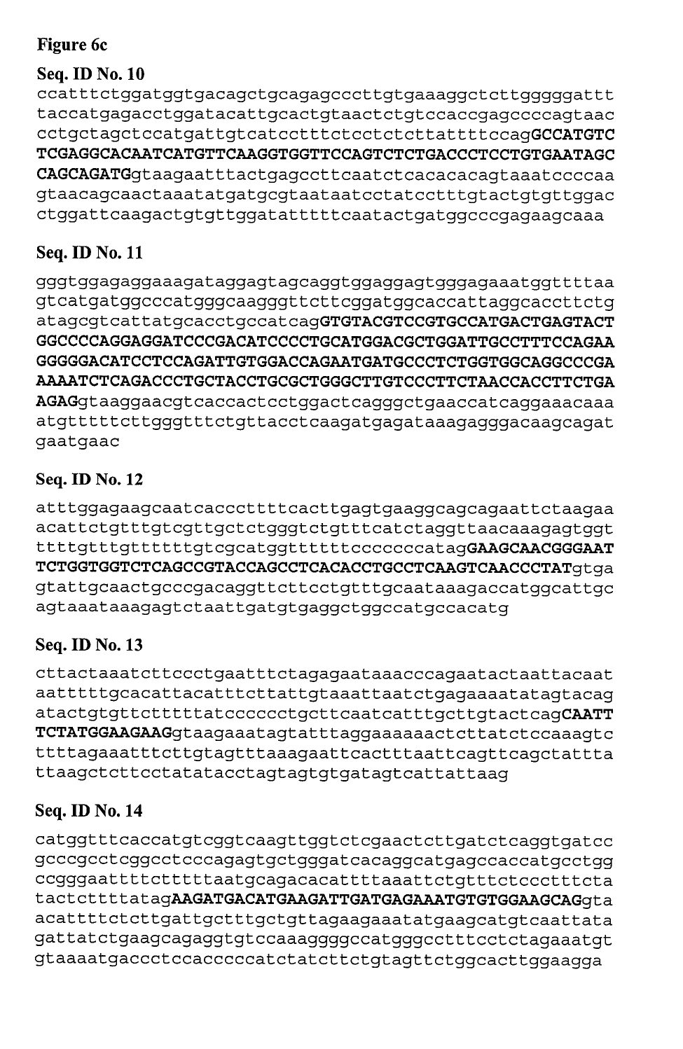

FIG. 6d Seq. ID Nos. 15-19. Shows the nucleotide sequence of the exon/intron organization of exons 14-18 of the MPP4 gene.

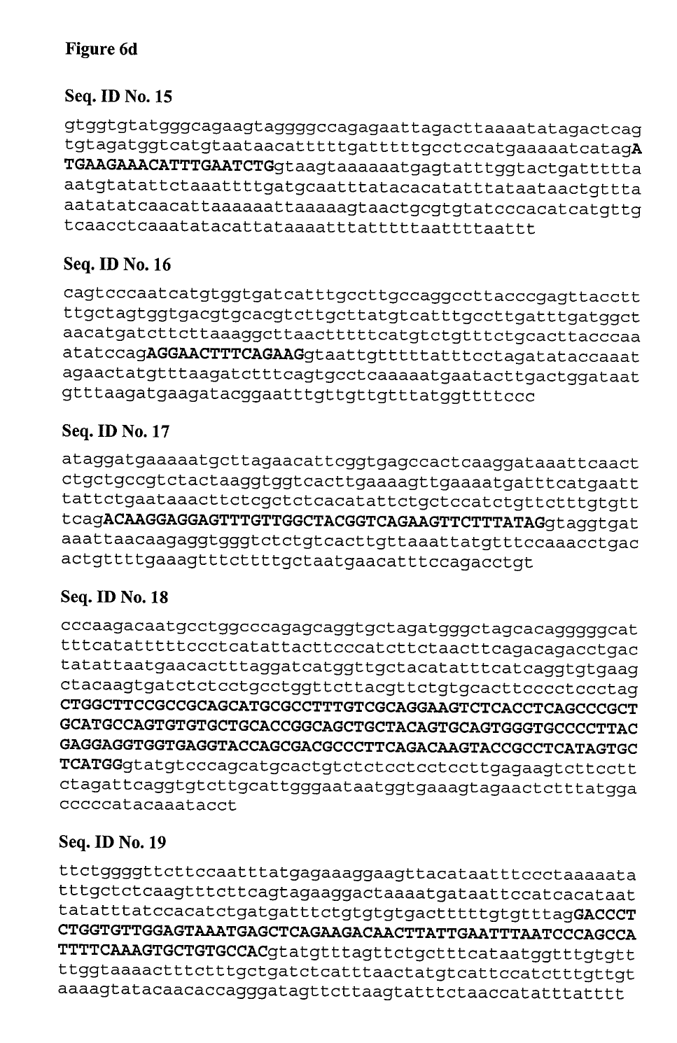

FIG. 6e Seq. ID Nos. 20-23. Shows the nucleotide sequence of the exon/intron organization of exons 19-22 of the MPP4 gene.

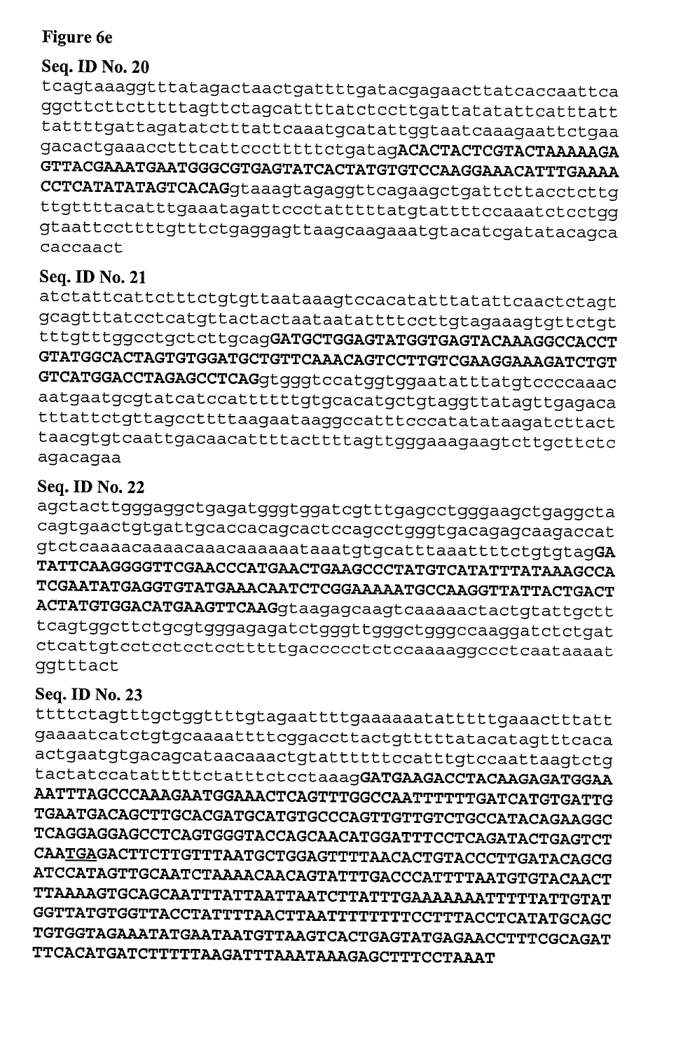

FIG. 7 Seq. ID Nos. 24 and 25. Shows the amino acid sequence of the predicted MPP4 protein; and the nucleotide sequence of the C7orf9 cDNA.

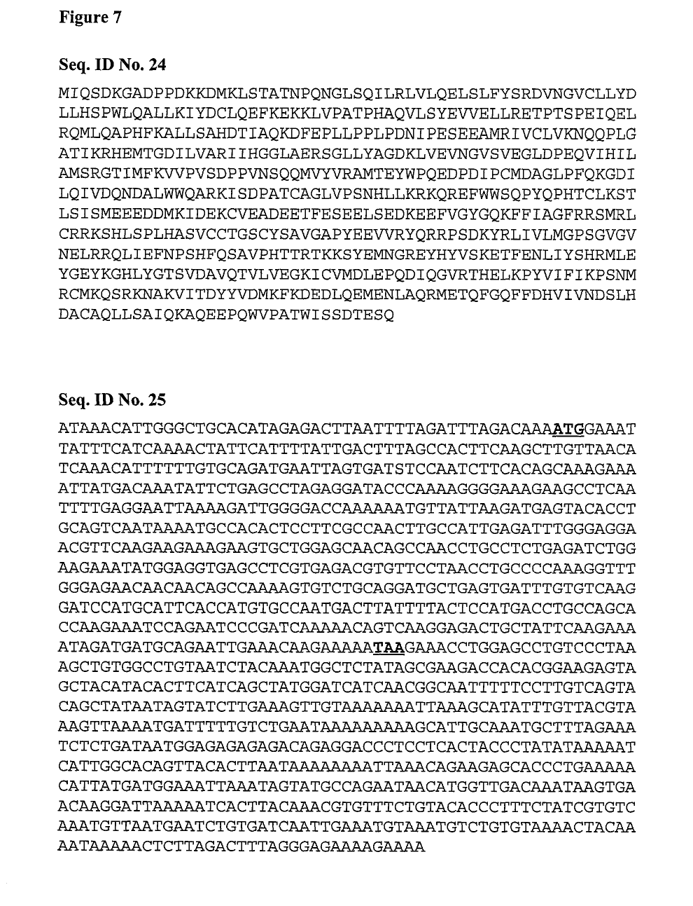

FIG. 8 Seq. ID Nos. 26-28. Shows the nucleotide sequence of the exon/intron organization of the C7orf9 gene;

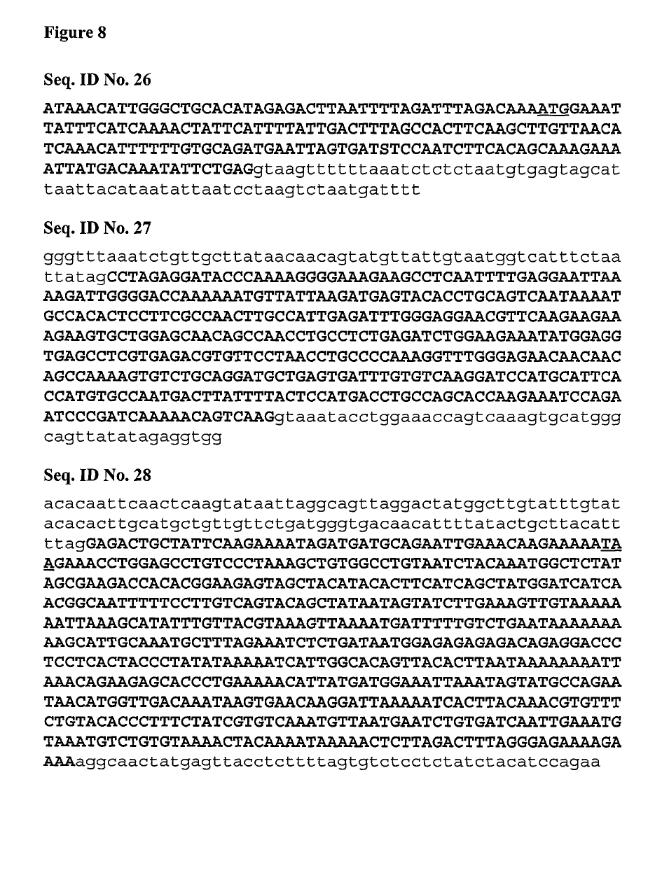

FIG. 9 Seq. ID Nos. 29-31. Shows the amino acid sequence of the predicted C7orf9 protein; shows the consensus nucleotide sequence of F379 cDNA; and shows the consensus amino acid sequence of the predicted F379 protein.

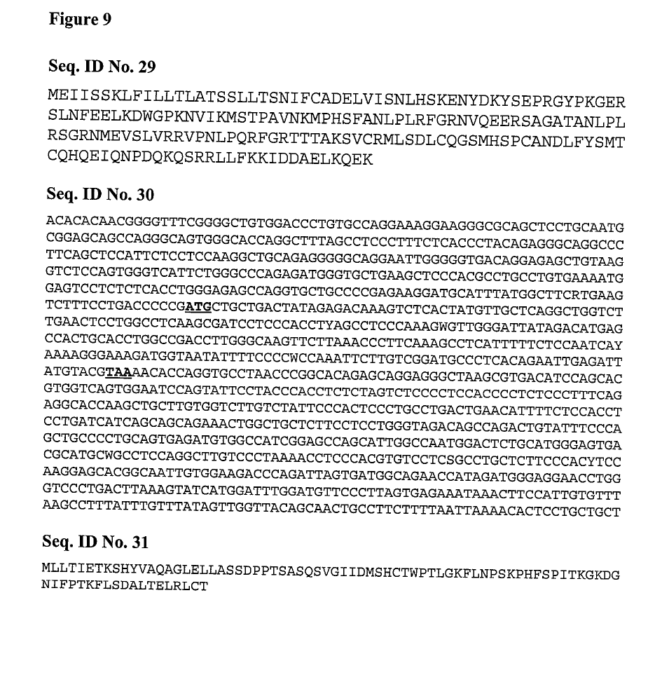

FIG. 10 Seq. ID Nos. 32-34. Shows the nucleotide sequence of the exon/intron organization of the F379 gene (based on the alignment to genomic clone RP11-395L14).

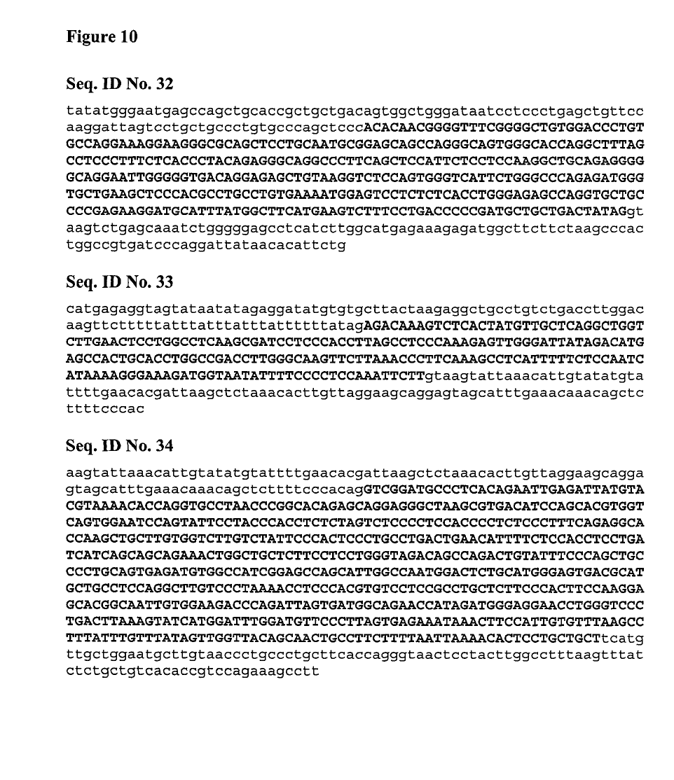

FIG. 11 Seq. ID Nos. 35-36. Shows the nucleotide sequence of C12orf7 cDNA variant 1; and the nucleotide sequence of C12orf7 cDNA variant 2;

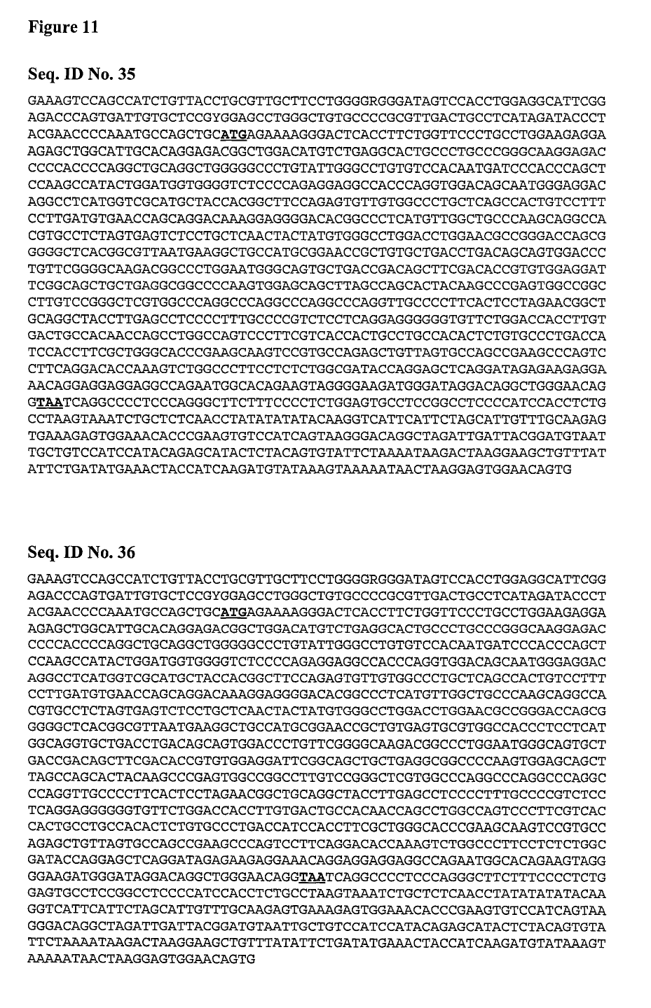

FIG. 12 Seq. ID Nos. 37-43. Shows the putative amino acid sequence of the C12orf7 protein (variant 1); and shows the putative amino acid sequence of the C12orf7 protein (variant 2); and shows the nucleotide sequence of the exon/intron organization of exons 1-4 variant 2 of the C12orf7 gene.

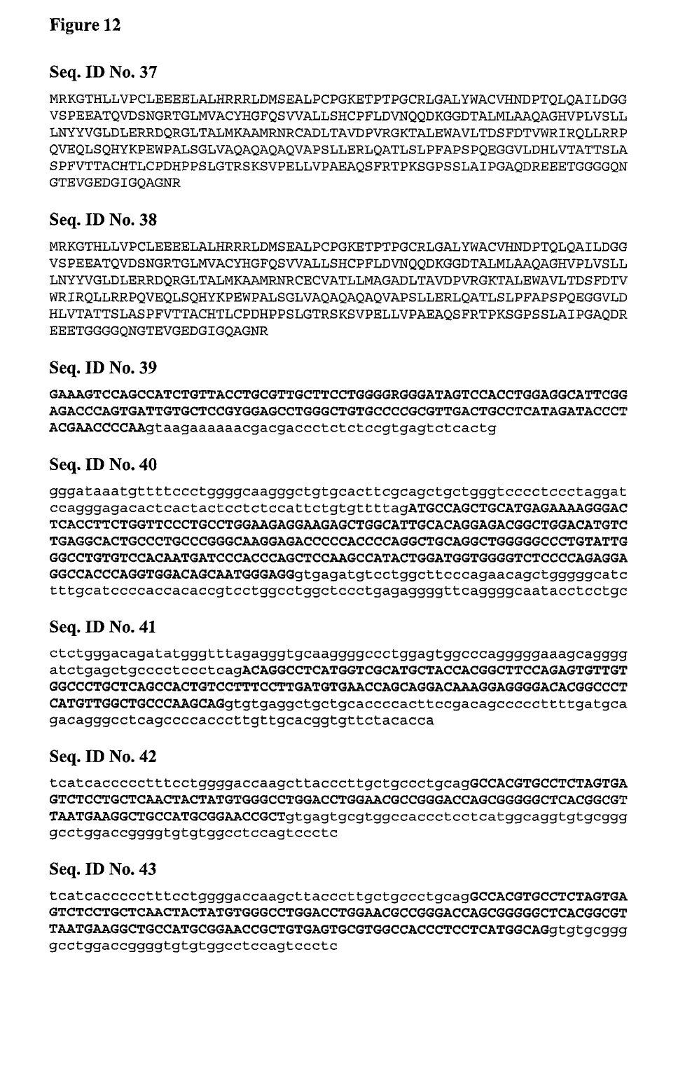

FIG. 13 Seq. ID Nos. 44 and 45. Shows the nucleotide sequence of the exon/intron organization of exons 5 and 6 of the C12orf7 gene.

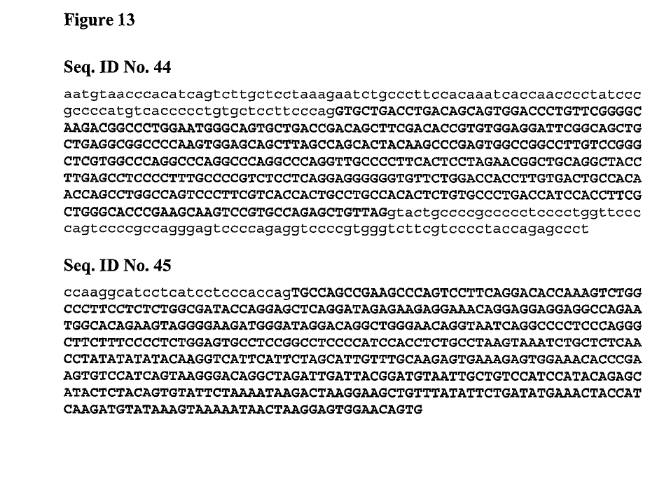

### DETAILED DESCRIPTION OF THE INVENTION

The present invention relates to an isolated nucleic acid molecule encoding the retina-specific human protein C7orf9, C12orf7, MPP4 or F379 or a protein exhibiting biological properties of C7orf9, C12orf7, MPP4 or F379 being selected from the group consisting of

(a) a nucleic acid molecule encoding a protein that comprises the amino acid sequence depicted in Seq. ID No. 24, 29, 31, 37 or 38;

(b) a nucleic acid molecule comprising the nucleotide sequence depicted in Seq. ID No. 1, 25, 30, 35 or 36;

(c) a nucleic acid molecule comprising the nucleotide sequence depicted in Seq. ID No. 2-23, 26-28, 32-34 or 39-45;

(d) a nucleic acid molecule which hybridizes to a nucleic acid molecule specified in (a) to (c);

(e) a nucleic acid molecule the nucleic acid sequence of which deviates from the nucleic sequences specified in (a) to (d) due to the degeneration of the genetic code; and

(f) a nucleic acid molecule, which represents a fragment, derivative or allelic variation of a nucleic acid sequence specified in (a) to (e).

As used herein, a protein exhibiting biological properties of C7orf9, C12orf7, MPP4 or F379 is understood to be a protein having at least one of the biological activities of C7orf9, C12orf7, MPP4 or F379.

As used herein, the term “isolated nucleic acid molecule” includes nucleic acid molecules substantially free of other nucleic acids, proteins, lipids, carbohydrates or other materials with which it is naturally associated. For example, an isolated nucleic acid molecule could be part of a vector or a composition of matter, or could be contained within a cell, and still be “isolated” because that vector, composition of matter, or particular cell is not the original environment of the nucleic acid molecule.

In a first embodiment, the invention provides an isolated nucleic acid molecule encoding the retina-specific human protein C7orf9, C12orf7, MPP4 or F379 comprising the amino acid sequence depicted in Se. ID No. 3, 6, 8, 11a or 11b. The present invention also provides a nucleic acid molecule comprising the nucleotide sequence depicted in Seq. ID No. 1, 25, 30, 35 or 36 (cDNA) or Seq. ID No. 2-23, 26-28, 32-34 or 39-45 (genomic DNA).

The nucleic acid molecules of the invention can be both DNA and RNA molecules. Suitable DNA molecules are, for example, genomic or cDNA molecules. It is understood that all nucleic acid molecules encoding all or a portion of C7orf9, C12orf7, MPP4 or F379 are also included, as long as they encode a protein with biological activity. The nucleic acid molecules of the invention can be isolated from natural sources or can be synthesized according to known methods.

The present invention also provides nucleic acid molecules which hybridize to the above nucleic acid molecules. As used herein, the term “hybridize” has the meaning of hybridization under conventional hybridization conditions, preferably under stringent conditions as described, for example, in Sambrook et al., Molecular Cloning, A Laboratory Manual, 2^{nd} edition (1989) Cold Spring Harbor Laboratory Press, Cold Spring Harbor, N.Y. Also contemplated are nucleic acid molecules that hybridize to the C7orf9, C12orf7, MPP4 or F379 nucleic acid molecules at lower stringency hybridization conditions. Changes in the stringency of hybridization and signal detection are primarily accomplished through the manipulation of formamide concentration (lower percentages of formamide result in lowered stringency), salt conditions, or temperature. For example, lower stringency conditions include an overnight incubation at 37° C. in a solution comprising 6×SSPE (20×SSPE=3M NaCl; 0.2M NaH₂PO₄; 0.02M EDTA, pH 7.4), 0.5% SDS, 30% formamide, 100 μg/ml salmon sperm blocking DNA, followed by washes at 50° C. with 1×SSPE, 0.1% SDS. In addition, to achieve even lower stringency, washes performed following stringent hybridization can be done at higher salt concentrations (e.g. 5×SSC). Variations in the above conditions may be accomplished through the inclusion and/or substitution of alternate blocking reagents used to suppress background in hybridization experiments. The inclusion of specific blocking reagents may require modification of the hybridization conditions described above, due to problems with compatibility.

Nucleic acid molecules that hybridize to the molecules of the invention can be isolated, e.g., from genomic or cDNA libraries that were produced from human cell lines or tissues. In order to identify and isolate such nucleic acid molecules the molecules of the invention or parts of these molecules or the reverse complements of these molecules can be used, for example by means of hybridization according to conventional methods (see, e.g., Sambrook et al., 1989, Molecular Cloning, A Laboratory Manual, 2^{nd} edition Cold Spring Harbor Laboratory Press, Cold Spring Harbor, N.Y.). As a hybridization probe nucleic acid molecules can be used, for example, that have exactly or basically the nucleotide sequence depicted in Seq. ID No. 1, 2-23, 25, 26-28, 30 and 32-34, respectively, or parts of these sequences. The fragments used as hybridization probe can be synthetic fragments that were produced by means of conventional synthesis methods and the sequence of which basically corresponds to the sequence of a nucleic acid molecule of the invention.

The nucleic acid molecules of the present invention also include molecules with sequences that are degenerate as a result of the genetic code.

In a further embodiment, the present invention provides nucleic acid molecules which comprise fragments, derivatives and allelic variants of the nucleic acid molecules described above encoding a protein of the invention. “Fragments” are understood to be parts of the nucleic acid molecules that are long enough to encode one of the described proteins. These fragments comprise nucleic acid molecules specifically hybridizing to transcripts of the nucleic acid molecules of the invention. These nucleic acid molecules can be used, for example, as probes or primers in the diagnostic assay and/or kit described below and, preferably, are oligonucleotides having a length of at least 15, preferably at least 50 nucleotides. The nucleic acid molecules and oligonucleotides of the invention can also be used, for example, as primers for a PCR reaction.

The term “derivative” in this context means that the sequences of these molecules differ from the sequences of the nucleic acid molecules described above at one or several positions but have a high level of homology to these sequences. Homology hereby means a sequence identity of at least 40%, in particular an identity of at least 60%, preferably of more than 80% and particularly preferred of more than 90%. These proteins encoded by the nucleic acid molecules have a sequence identity to the amino acid sequence depicted in Seq. ID No. 24, 29 and 31, respectively, of at least 80%, preferably of 85% and particularly preferred of more than 90%, 95%, 97% and 99%. The deviations to the above-described nucleic acid molecules may have been produced by deletion, substitution, insertion or recombination.

The nucleic acid molecules that are homologous to the above-described molecules and that represent derivatives of these molecules usually are variations of these molecules that represent modifications having the same biological function. They can be naturally occurring variations, for example sequences from other organisms, or mutations that can either occur naturally or that have been introduced by specific mutagenesis. Furthermore, the variations can be synthetically produced sequences. The allelic variants can be either naturally occurring variants or synthetically produced variants or variants produced by recombinant DNA processes.

Generally, by means of conventional molecular biological processes it is possible (see, e.g., Sambrook et al., 1989, Molecular Cloning, A Laboratory Manual, 2^{nd} edition Cold Spring Harbor Laboratory Press, Cold Spring Harbor, N.Y.) to introduce different mutations into the nucleic acid molecules of the invention. As a result C7orf9, C12orf7, MPP4 or F379 proteins or C7orf9, C12orf7, MPP4 or F379 related proteins with possibly modified biological properties are synthesized. One possibility is the production of deletion mutants in which nucleic acid molecules are produced by continuous deletions from the 5′- or 3′-terminal of the coding DNA sequence and that lead to the synthesis of proteins that are shortened accordingly. Another possibility is the introduction of single-point mutation at positions where a modification of the amino acid sequence influences, e.g., the enzyme activity or the regulation of the enzyme. By this method muteins can be produced, for example, that possess a modified Kₘ-value or that are no longer subject to the regulation mechanisms that normally exist in the cell, e.g. with regard to allosteric regulation or covalent modification. Such muteins might also be valuable as therapeutically useful inhibitors (antagonists) of C7orf9, C12orf7, MPP4 and F379, respectively.

For the manipulation in prokaryotic cells by means of genetic engineering the nucleic acid molecules of the invention or parts of these molecules can be introduced into plasmids allowing a mutagenesis or a modification of the sequence by recombination of DNA sequences. By means of conventional methods (cf. Sambrook et al., 1989, Molecular Cloning: A Laboratory Manual, 2^{nd} edition, Cold Spring Harbor Laboratory Press, N.Y., USA) bases can be exchanged and natural or synthetic sequences can be added. In order to link the DNA fragments with each other adapters or linkers can be added to the fragments. Furthermore, manipulations can be performed that provide suitable cleavage sites or that remove superfluous DNA or cleavage sites. If insertions, deletions or substitutions are possible, in vitro mutagenesis, primer repair, restriction or ligation can be performed. As analysis method usually sequence analysis, restriction analysis and other biochemical or molecular biological methods are used.

The proteins encoded by the various variants of the nucleic acid molecules of the invention show certain common characteristics, such as enzyme activity, molecular weight, immunological reactivity or conformation or physical properties like the electrophoretical mobility, chromatographic behavior, sedimentation coefficients, solubility, spectroscopic properties, stability; pH optimum, temperature optimum.

The invention furthermore relates to vectors containing the nucleic acid molecules of the invention. Preferably, they are plasmids, cosmids, viruses, bacteriophages and other vectors usually used in the field of genetic engineering. Vectors suitable for use in the present invention include, but are not limited to the T7-based expression vector for expression in bacteria, the pMSXND expression vector for expression in mammalian cells and baculovirus-derived vectors for expression in insect cells. Preferably, the nucleic acid molecule of the invention is operatively linked to the regulatory elements in the recombinant vector of the invention that guarantee the transcription and synthesis of an RNA in prokaryotic and/or eukaryotic cells that can be translated. The nucleotide sequence to be transcribed can be operably linked to a promoter like a T7, metallothionein I or polyhedrin promoter.

In a further embodiment, the present invention relates to recombinant host cells transiently or stably containing the nucleic acid molecules or vectors of the invention. A host cell is understood to be an organism that is capable to take up in vitro recombinant DNA and, if the case may be, to synthesize the proteins encoded by the nucleic acid molecules of the invention. Preferably, these cells are prokaryotic or eukaryotic cells, for example mammalian cells, bacterial cells, insect cells or yeast cells. The host cells of the invention are preferably characterized by the fact that the introduced nucleic acid molecule of the invention either is heterologous with regard to the transformed cell, i.e. that it does not naturally occur in these cells, or is localized at a place in the genome different from that of the corresponding naturally occurring sequence.

A further embodiment of the invention relates to isolated proteins exhibiting biological properties of the human retina-specific proteins C7orf9, C12orf7, MPP4 or F379 and being encoded by the nucleic acid molecules of the invention, as well as to methods for their production, whereby, e.g, a host cell of the invention is cultivated under conditions allowing the synthesis of the protein and the protein is subsequently isolated from the cultivated cells and/or the culture medium. Isolation and purification of the recombinantly produced proteins may be carried out by conventional means including preparative chromatography and affinity and immunological separations involving affinity chromatography with monoclonal or polyclonal antibodies, e.g. with an anti-C7orf9-, anti-MPP4-, anti-C12orf7-, and anti-F379-antibody, respectively.

As used herein, the term “isolated protein” includes proteins substantially free of other proteins, nucleic acids, lipids, carbohydrates or other materials with which it is naturally associated. Such proteins however not only comprise recombinantly produced proteins but include isolated naturally occurring proteins, synthetically produced proteins, or proteins produced by a combination of these methods. Means for preparing such proteins are well understood in the art. The proteins of the invention are preferably in a substantially purified form. A recombinantly produced version of a C7orf9, C12orf7, MPP4 or F379 protein, including the secreted protein, can be substantially purified by the one-step method described in Smith and Johnson, Gene 67:31-40 (1988).

In a further preferred embodiment, the invention relates to nucleic acid molecules of at least 15 nucleotides in length hybridizing specifically with a nucleic acid molecule as described above or with a complementary strand thereof. Specific hybridization occurs preferably under stringent conditions and implies no or very little cross-hybridization with nucleotide sequences encoding no or substantially different proteins. Such nucleic acid molecules may be used as probes and/or for the control of gene expression. Nucleic acid probe technology is well known to those skilled in the art who will readily appreciate that such probes may vary in length. Preferred are nucleic acid probes of 17 to 35 nucleotides in length. Of course, it may also be appropriate to use nucleic acids of up to 100 and more nucleotides in length. The nucleic acid probes of the invention are useful for various applications. On the one hand, they may be used as PCR primers for amplification of nucleic acid molecules according to the invention or for detecting mutations within said nucleic acid molecules. Another application is the use as a hybridization probe to identify polynucleotides hybridizing to the nucleic acid molecules of the invention by homology screening of genomic DNA libraries. Nucleic acid molecules according to this preferred embodiment of the invention which are complementary to a nucleic acid molecule as described above may also be used for repression of expression of a gene comprising such a nucleic acid molecule, for example due to an antisense or triple helix effect or for the construction of appropriate ribozymes (see, e.g., EP-B1 0 291 533, EP-A1 0 321 201, EP-A2 0 360 257) which specifically cleave the (pre)-mRNA of a gene comprising a nucleic acid molecule of the invention. Selection of appropriate target sites and corresponding ribozymes can be done as described for example in Steinecke, Ribozymes, Methods in Cell Biology 50, Galbraith et al. eds Academic Press, Inc. (1995), 449-460. Standard methods relating to antisense technology have also been described (Melani, Cancer Res. 51 (1991), 2897-2901). Said nucleic acid molecules may be chemically synthesized or transcribed by an appropriate vector containing a chimeric gene which allows for the transcription of said nucleic acid molecule in the cell. Such nucleic acid molecules may further contain ribozyme sequences as described above.

Thus, the present invention also relates to (i) an antisense RNA sequence characterized in that it is complementary to an mRNA transcribed from a nucleic acid molecule of the present invention or a part thereof and can selectively bind to said mRNA, said sequence being capable of inhibiting the synthesis of the protein encoded by said nucleic acid molecules, and (ii) a ribozyme characterized in that it is complementary to an mRNA transcribed from a nucleic acid molecule of the present invention or a part thereof and can selectively bind to and cleave said mRNA, thus inhibiting the synthesis of the proteins encoded by said nucleic acid molecules. Preferably, the antisense RNA and ribozyme of the invention are complementary to the coding region of the mRNA, e.g. to the 5′ part of the coding region. The person skilled in the art provided with the sequences of the nucleic acid molecules of the present invention will be in a position to produce and utilize the above described antisense RNAs or ribozymes.

It is also to be understood that the nucleic acid molecules of the invention can be used for “gene targeting” and/or “gene replacement”, for restoring a mutant gene or for creating a mutant gene via homologous recombination; see for example Mouellic, PNAS USA 87 (1990), 4712-4716; Joyner, Gene Targeting, A Practical Approach, Oxford University Press.

Furthermore, the person skilled in the art is well aware that it is also possible to label such a nucleic acid probe with an appropriate marker for specific applications, such as for the detection of the presence of a nucleic acid molecule of the invention in a sample derived from an organism, in particular mammals, preferably human. A number of companies such as Pharmacia Biotech (Piscataway, N.J.), Promega (Madison, Wis.), and US Biochemical Corp (Cleveland, Ohio) supply commercial kits and protocols for these procedures. Suitable reporter molecules or labels include those radionuclides, enzymes, fluorescent, chemoluminescent, or chromogenic agents as well as substrates, cofactors, inhibitors, magnetic particles and the like. Patents teaching the use of such labels include U.S. Pat. Nos. 3,817,837; 3,850,752; 3,939,350; 3,996,345; 4,227,437; 4,275,149 and 4,366,241. Also, recombinant immunoglobulins may be produced as shown in 4,816,567 incorporated herein by reference.

Furthermore, the so-called “peptide nucleic acid” (PNA) technique can be used for the detection or inhibition of the expression of a nucleic acid molecule of the invention. For example, the binding of PNAs to complementary as well as various single stranded RNA and DNA nucleic acid molecules can be systematically investigated using thermal denaturation and BIAcore surface-interaction techniques (Jensen, Biochemistry 36 (1997), 5072-5077). Furthermore, the nucleic acid molecules described above as well as PNAs derived therefrom can be used for detecting point mutations by hybridization with nucleic acids obtained from a sample with an affinity sensor, such as BIAcore; see Gotoh, Rinsho Byori 45 (1997), 224-228. Hybridization based DNA screening on peptide nucleic acids (PNA) oligomer arrays are described in the prior art, for example in Weiler, Nucleic Acids Research 25 (1997), 2792-2799. The synthesis of PNAs can be performed according to methods known in the art, for example, as described in Koch, J. Pept. Res. 49 (1997), 80-88; Finn, Nucleic Acids Research 24 (1996), 3357-3363. Further possible applications of such PNAs, for example as restriction enzymes or as templates for the synthesis of nucleic acid oligonucleotides are known to the person skilled in the art and are, for example, described in Veselkov, Nature 379 (1996), 214 and Bohler, Nature 376 (1995), 578-581.

In still a further embodiment, the present invention relates to inhibitors of C7orf9, C12orf7, MPP4 or F379 which fulfill a similar purpose as the antisense RNAs or ribozymes mentioned above, i.e. reduction or elimination of biologically active C7orf9, C12orf7, MPP4 or F379 molecules. Such inhibitors can be, for instance, structural analogues of the corresponding protein or muteins that act as antagonists. In addition, such inhibitors comprise molecules identified by the use of the recombinantly produced proteins, e.g. the recombinantly produced protein can be used to screen for and identify inhibitors, for example, by exploiting the capability of potential inhibitors to bind to the protein under appropriate conditions. The inhibitors can, for example, be identified by preparing a test mixture wherein the inhibitor candidate is incubated with the protein C7orf9, C12orf7, MPP4 or F379 under appropriate conditions that allow C7orf9, C12orf7, MPP4 or F379 to be in a native conformation. Such an in vitro test system can be established according to methods well known in the art. Inhibitors can be identified, for example, by first screening for either synthetic or naturally occurring molecules that bind to the recombinantly produced C7orf9, C12orf7, MPP4 or F379 protein and then, in a second step, by testing those selected molecules in cellular assays for inhibition of the C7orf9, C12orf7, MPP4 or F379 protein, as reflected by inhibition of at least one of the biological activities. Such screening for molecules that bind the C7orf9, C12orf7, MPP4 or F379 protein could easily performed on a large scale, e.g. by screening candidate molecules from libraries of synthetic and/or natural molecules. Such an inhibitor is, e.g., a synthetic organic chemical, a natural fermentation product, a substance extracted from a microorganism, plant or animal, or a peptide. Additional examples of inhibitors are specific antibodies, preferably monoclonal antibodies. Moreover, the nucleic sequences of the invention and the encoded proteins can be used to identify further factors involved in development and progression of macular degeneration. The proteins of the invention can, e.g., be used to identify further (unrelated) proteins which are associated with macular degeneration using screening methods based on protein/protein interactions, e.g. the two-hybrid-system.

It can be expected that macular degeneration, e.g. AMD, is due to (i) aberrant expression of the gene(s) encoding C7orf9, C12orf7, MPP4 and/or F379, (ii) mutations within the gene(s) encoding C7orf9, C12orf7, MPP4 and/or F379 leading to the production of proteins showing reduced or eliminated biological activity or (iii) differences in the chromosomal location due to translocation, inversion etc. Thus, the nucleic acid molecules of the invention are also useful in numerous ways as reagents for detecting the above differences, e.g. by comparing the results obtained with normal individuals and the results obtained with affected individuals (or carriers of the disease).

Thus, the present invention also provides a method for diagnosing macular degeneration or a predisposition for macular degeneration, preferably AMD, which comprises contacting a target sample suspected to contain the retina-specific human protein C7orf9, C12orf7, MPP4 and/or F379 or the C7orf9, C12orf7, MPP4 and/or F379 encoding nucleic acid with a reagent which reacts with C7orf9, C12orf7, MPP4 and/or F379 and/or C7orf9, C12orf7, MPP4 and/or F379 encoding nucleic acid and detecting the C7orf9, C12orf7, MPP4 and/or F379 protein and/or C7orf9, C12orf7, MPP4 and/or F379 encoding nucleic acid, wherein the presence of a mutation within the C7orf9, C12orf7, MPP4 and/or F379 encoding nucleic acid, a chromosomal rearrangement or abnormal levels of the C7orf9, C12orf7, MPP4 and/or F379 protein and/or C7orf9, C12orf7, MPP4 and/or F379 encoding mRNA are indicative for macular degeneration or a predisposition for macular degeneration.

The target cellular component, e.g. C7orf9, C12orf7, MPP4 and/or F379 encoding nucleic acid, e.g., in biological fluids or tissues, may be detected directly in situ, e.g. by in situ hybridization or it may be isolated from other cell components by common methods known to those skilled in the art before contacting with a probe. Detection methods include Northern blot analysis, RNase protection, in situ methods, e.g. in situ hybridization, in vitro amplification methods (PCR RT-PCR, LCR, QRNA replicase or RNA-transcription/amplification (TAS, 3SR), reverse dot blot disclosed in EP-B1 0 237 362)), immunoassays, Western blot and other detection assays that are known to those skilled in the art. Products obtained by in vitro amplification can be detected according to established methods, e.g. by separating the products on agarose gels and by subsequent staining with ethidium bromide. Alternatively, the amplified products can be detected by using labeled primers for amplification or labeled dNTPs.

Sequences can be mapped to chromosomes by preparing PCR primers (preferably 15-25 bp) from the sequences shown in Seq. ID No. 1, 2-23, 25, 26-28, 30, 32-34, 35, 36 or 39-45. Primers can be selected using computer analysis so that primers do not span more than one predicted exon in the genomic DNA. These primers are then used for PCR screening of somatic cell hybrids containing individual human chromosomes. Only those hybrids containing the human C7orf9, C12orf7, MPP4 or F379 nucleic acid molecule(s) will yield an amplified fragment. Similarly, somatic hybrids provide a rapid method of PCR mapping the polynucleotides to particular chromosomes. Three or more clones can be assigned per day using a single thermal cycler. Moreover, sublocalization of the C7orf9, C12orf7, MPP4 or F379 genes can be achieved with panels of specific chromosome fragments. Other gene mapping strategies that can be used include in situ hybridization, prescreening with labeled flow-sorted chromosomes, and preselection by hybridization to construct chromosome specific cDNA libraries. Precise chromosomal location of the C7orf9, C12orf7, MPP4 or F379 genes can also be achieved using fluorescence in situ hybridization (FISH) of a metaphase chromosomal spread. This technique uses polynucleotides as short as 500 or 600 bases; however, polynucleotides 1,000-4,000 bp are preferred. For a review of this technique, see Verma et al., “Human Chromosomes: a Manual of Basic Techniques,” Pergamon Press, New York (1988). For chromosome mapping, the nucleic acid molecules of the invention can be used individually (to mark a single chromosome or a single site on that chromosome) or in panels (for marking multiple sites and/or multiple chromosomes). Preferred nucleic acid molecules correspond to the noncoding regions of the cDNAs because the coding sequences are more likely conserved within gene families, thus increasing the chance of cross hybridization during chromosomal mapping. Once a gene has been mapped to a precise chromosomal location, the physical position of the gene can be used in linkage analysis. Linkage analysis establishes co-inheritance between a chromosomal location and presentation of the disease. Thus, once co-inheritance is established, differences in the C7orf9, C12orf7, MPP4 and/or F379 gene(s) and the corresponding gene(s) between affected and unaffected individuals can be examined. First, visible structural alterations in the chromosomes, such as deletions or translocations, are examined in chromosome spreads or by PCR. If no structural alterations exist, the presence of point mutations are ascertained. Mutations observed in some or all affected individuals, but not in normal individuals, indicate that the mutation may cause the disease. However, complete sequencing of the C7orf9, C12orf7, MPP4 or F379 polypeptide and the corresponding gene from several normal individuals might be required to distinguish the mutation from a polymorphism. If a new polymorphism is identified, this polymorphic polypeptide can be used for further linkage analysis.

Furthermore, increased or decreased expression of the gene in affected individuals as compared to unaffected individuals can be assessed using the nucleic acid molecules of the invention. Expression of C7orf9, C12orf7, MPP4 and F379, respectively, in retinal tissues can be studied with classical immunohistological methods (Jalkanen et al., J. Cell. Biol. 101 (1985), 976-985; Jalkanen et al., J. Cell. Biol. 105 (1987), 3087-3096; Sobol et al. Clin. Immunpathol. 24 (1982), 139-144; Sobol et al., Cancer 65 (1985), 2005-2010). Other antibody based methods useful for detecting protein gene expression include immunoassays, such as the enzyme-linked immunosorbent assay (ELISA) and the radioimmunoassay (RIA). Suitable antibody assay labels are known in the art and include enzyme labels, such as, glucose oxidase, and radioisotopes, such as iodine (¹²⁵I, ¹²¹I), carbon (¹⁴C), sulfur (³⁵S), tritium (³H), indium (¹¹²In), and technetium (⁹⁹mTc), and fluorescent labels, such as fluorescein and rhodamine, and biotin. In addition to assaying C7orf9, C12orf7, MPP4 and F379 in a biological sample, the protein can also be detected in vivo by imaging. Antibody labels or markers for in vivo imaging of protein include those detectable by X-radiography, NMR or ESR. For X-radiography, suitable labels include radioisotopes such as barium or cesium, which emit detectable radiation but are not overtly harmful to the subject. Suitable markers for NMR and ESR include those with a detectable characteristic spin, such as deuterium, which may be incorporated into the antibody by labeling of nutrients for the relevant hybridoma. A protein-specific antibody or antibody fragment which has been labeled with an appropriate detectable imaging moiety, such as a radioisotope (for example, ¹³¹I, ¹¹²In, ⁹⁹mTc), a radio-opaque substance, or a material detectable by nuclear magnetic resonance, is introduced (for example, parenterally, subcutaneously, or intraperitoneally) into the mammal. It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries of ⁹⁹mTc. The labeled antibody or antibody fragment will then preferentially accumulate at the location of cells which contain the specific protein.

The concentration of the C7orf9, C12orf7, MPP4 and/or F379 protein can also be diagnostically relevant. When the target is the protein, the reagent is typically an anti-C7orf9-, anti-C12orf7-, anti-MPP4 or anti-F379-antibody probe. The term “antibody”, preferably, relates to antibodies which consist essentially of pooled monoclonal antibodies with different epitopic specificities, as well as distinct monoclonal antibody preparations. Monoclonal antibodies are made from an antigen containing a fragment of the proteins of the invention by methods well known to those skilled in the art (see, e.g., Kohler et al., Nature 256 (1975), 495). As used herein, the term “antibody” (Ab) or “monoclonal antibody” (Mab) is meant to include intact molecules as well as antibody fragments (such as, for example, Fab and F(ab′)2 fragments) which are capable of specifically binding to the protein. Fab and F(ab′)2 fragments lack the Fc fragment of intact antibody, clear more rapidly from the circulation, and may have less non-specific tissue binding than an intact antibody. (Wahl et al., J. Nucl. Med. 24:316-325 (1983).) Thus, these fragments are preferred, as well as the products of a FAB or other immunoglobulin expression library. Moreover, antibodies of the present invention include chimerical, single chain, and humanized antibodies.

The probes can be detectably labeled, for example, with a radioisotope, a bioluminescent compound, a chemoluminescent compound, a fluorescent compound, a metal chelate, or an enzyme. A variety of techniques are available for labeling biomolecules, are well known to the person skilled in the art and are considered to be within the scope of the present invention. Such techniques are, e.g., described in Tijssen, “Practice and theory of enzyme immuno assays”, Burden, R H and von Knippenburg (Eds), Volume 15 (1985), “Basic methods in molecular biology”; Davis L G, Dibmer M D; Battey Elsevier (1990), Mayer et al., (Eds) “Immunochemical methods in cell and molecular biology” Academic Press, London (1987), or in the series “Methods in Enzymology”, Academic Press, Inc. There are many different labels and methods of labeling known to those of ordinary skill in the art. Commonly used labels comprise, inter alia, fluorochromes (like fluorescein, rhodamine, Texas Red, etc.), enzymes (like horse radish peroxidase, beta-galactosidase, alkaline phosphatase), radioactive isotopes (like ³²P or ¹²⁵I), biotin, digoxygenin, colloidal metals, chemo- or bioluminescent compounds (like dioxetanes, luminol or acridiniums). Labeling procedures, like covalent coupling of enzymes or biotinyl groups, iodinations, phosphorylations, biotinylations, random priming, nick-translations, tailing (using terminal transferases) are well known in the art. Detection methods comprise, but are not limited to, autoradiography, fluorescence microscopy, direct and indirect enzymatic reactions, etc.

Any of the above described alterations (altered expression, chromosomal rearrangement, or mutation) can be used as a diagnostic or prognostic marker.

The present invention also relates to a method for treating macular degeneration or a predisposition for macular degeneration, preferably AMD, which comprises administering to a mammalian subject a therapeutically effective amount of a reagent which decreases, inhibits or increases expression of C7orf9, C12orf7, MPP4 and/or F379 or which leads to the expression of biologically active C7orf9, C12orf7, MPP4 and/or F379 protein. This method also comprises a prenatal diagnosis.

Examples of such reagents are the nucleic acid molecules of the invention, the above described antisense RNAs, ribozymes or inhibitors, e.g. specific antibodies. For example, administration of an antibody directed to the protein can bind and reduce overproduction of the protein.

Thus, the nucleic acid molecules can be used to control gene expression through triple helix formation or antisense DNA or RNA. Both methods rely on binding of the nucleic acid molecule to DNA or RNA. For these techniques, preferred polynucleotides are usually 20 to 40 bases in length and complementary to either the region of the gene involved in transcription (triple helix-see Lee, Nucl. Acids Res. 6 (1979), 3073; Cooney, Science 241 (1988), 456; and Dervan, Science 251 (1991), 1360) or to the mRNA itself (antisense—Okano, J. Neurochem. 56 (1991), 560; Oligodeoxy-nucleotides as Antisense Inhibitors of Gene Expression, CRC Press, Boca Raton, Fla. (1988).) Triple helix formation optimally results in a shut-off of RNA transcription from DNA, while antisense RNA hybridization blocks translation of an mRNA molecule into polypeptide. Both techniques are effective in model systems, and the information disclosed herein can be used to design antisense or triple helix polynucleotides in an effort to treat disease. Additionally, a decrease or inhibition of gene expression can be achieved by using the above discussed ribozymes or by making dominant-negative mutants of C7orf9, C12orf7, MPP4 and/or F379 by gene therapy to inhibit C7orf9, C12orf7, MPP4 and/or F379 function in disease. Finally, if macular degeneration is due to over-expression of C7orf9, C12orf7, MPP4 and/or F379 an inhibitor of the C7orf9, C12orf7, MPP4 and/or F379 protein as discussed above, e.g. an anti-C7orf9-, an anti-C12orf7-, anti-MPP4- or anti-F379-antibody can be administered. Such an antibody can bind and reduce overproduction of the protein.

In cases where the disease is due to a decreased expression of C7orf9, C12orf7, MPP4 and/or F379 a therapeutic effect can be obtained by administering the nucleic acid molecule(s) encoding C7orf9, C12orf7, MPP4 and/or F379 or the protein(s) itself.

The nucleic acid molecules of the invention are also useful in gene therapy. One goal of gene therapy is to insert a normal gene into an organism having a defective gene, in an effort to correct the genetic defect. The nucleic acid molecules of the invention offer a means of targeting such genetic defects in a highly accurate manner. Another goal is to insert a new gene that was not present in the host genome, thereby producing a new trait in the host cell.

For administration, the above reagents are preferably combined with suitable pharmaceutical carriers. Examples of suitable pharmaceutical carriers are well known in the art and include phosphate buffered saline solutions, water, emulsions, such as oil/water emulsions, various types of wetting agents, sterile solutions etc. Such carriers can be formulated by conventional methods and can be administered to the subject at a suitable dose. Administration of the suitable compositions may be effected by different ways, e.g. by intravenous, intraperetoneal, subcutaneous, intramuscular, topical or intradermal administration. The route of administration, of course, depends, e.g., an the kind of compound contained in the pharmaceutical composition. The dosage regimen will be determined by the attending physician and other clinical factors. As is well known in the medical arts, dosages for any one patient depends on many factors, including the patients size, body surface area, age, sex, the particular compound to be administered, time and route of administration, the kind and stage of the disease, general health and other drugs being administered concurrently.

The delivery of the nucleic acid molecules of the invention, antisense RNAs or ribozymes of the invention can be achieved by direct application or, preferably, by using a recombinant expression vector such as a chimeric virus containing these compounds or a colloidal dispersion system. By delivering these nucleic acids to the desired target, the intracellular expression of C7orf9, C12orf7, MPP4 and/or F379 and, thus, the level of C7orf9, C12orf7, MPP4 and/or F379 can be increased or decreased.

Direct application to the target site can be performed, e.g., by ballistic delivery, as a colloidal dispersion system or by catheter to a site in artery. The colloidal dispersion systems which can be used for delivery of the above nucleic acids include macromolecule complexes, nanocapsules, microspheres, beads and lipid-based systems including oil-in-water emulsions, (mixed) micelles, liposomes and lipoplexes. The preferred colloidal system is a liposome. The composition of the liposome is usually a combination of phospholipids and steroids, especially cholesterol. The skilled person is in a position to select such liposomes which are suitable for the delivery of the desired nucleic acid molecule. Organ-specific or cell-specific liposomes can be used in order to achieve delivery only to the retinal tissue. The targeting of liposomes can be carried out by the person skilled in the art by applying commonly known methods. This targeting includes passive targeting (utilizing the natural tendency of the liposomes to distribute to cells of the RES in organs which contain sinusoidal capillaries) or active targeting (for example by coupling the liposome to a specific ligand, e.g., an antibody, a receptor, sugar, glycolipid, protein etc., by well known methods). In the present invention monoclonal antibodies are preferably used to target liposomes to specific tumors via specific cell-surface ligands.

Preferred recombinant vectors useful for gene therapy are viral vectors, e.g. adenovirus, herpes virus, vaccinia, or, more preferably, an RNA virus such as a retrovirus. Even more preferably, the retroviral vector is a derivative of a murine or avian retrovirus. Examples of such retroviral vectors which can be used in the present invention are: Moloney murine leukemia virus (MoMuLV), Harvey murine sarcoma virus (HaMuSV), murine mammary tumor virus (MuMTV) and Rous sarcoma virus (RSV). Most preferably, a non-human primate retroviral vector is employed, such as the gibbon ape leukemia virus (GaLV), providing a broader host range compared to murine vectors. Since recombinant retroviruses are defective, assistance is required in order to produce infectious particles. Such assistance can be provided, e.g., by using helper cell lines that contain plasmids encoding all of the structural genes of the retrovirus under the control of regulatory sequences within the LTR. Suitable helper cell lines are well known to those skilled in the art. Said vectors can additionally contain a gene encoding a selectable marker so that the transduced cells can be identified. Moreover, the retroviral vectors can be modified in such a way that they become target specific. This can be achieved, e.g., by inserting a polynucleotide encoding a sugar, a glycolipid, or a protein, preferably an antibody. Those skilled in the art know additional methods for generating target specific vectors. Further suitable vectors and methods for in vitro- or in vivo-gene therapy are described in the literature and are known to the persons skilled in the art; see, e.g., WO 94/29469 or WO 97/00957.

In order to achieve expression only in the target organ, the nucleic acids encoding, e.g. an antisense RNA or ribozyme can also be operably linked to a tissue specific promoter and used for gene therapy. Such promoters are well known to those skilled in the art (see e.g. Zimmermann et al, (1994) Neuron 12, 11-24; Vidal et al., (1990) EMBO J. 9, 833-840; Mayford et al., (1995), Cell 81, 891-904; Pinkert et al., (1987) Genes & Dev. 1, 268-76).

For use in the diagnostic research, kits are also provided by the present invention. Such kits are useful for the detection of macular degeneration or a predisposition for macular degeneration and comprise at least one of the aforementioned nucleic acid molecules, vectors, proteins, antibodies or compounds and optionally suitable means for detection.

In this embodiment, the nucleic acid molecules, proteins, antibodies or compounds identified above are preferably detectably labeled as already described above.

In addition, the above-described compounds etc. may be attached to a solid phase. Solid phases are known to those in the art and may comprise polystyrene beads, latex beads, magnetic beads, colloid metal particles, glass and/or silicon chips and surfaces, nitrocellulose strips, membranes, sheets, animal red blood cells, or red blood cell ghosts, duracytes and the walls of wells of a reaction tray, plastic tubes or other test tubes. Suitable methods of immobilizing nucleic acids, (poly)peptides, proteins, antibodies, etc. on solid phases include but are not limited to ionic, hydrophobic, covalent interactions and the like. The solid phase can retain one or more additional receptor(s) which has/have the ability to attract and immobilize the region as defined above. This receptor can comprise a charged substance that is oppositely charged with respect to the reagent itself or to a charged substance conjugated to the capture reagent or the receptor can be any specific binding partner which is immobilized upon (attached to) the solid phase and which is able to immobilize the reagent as defined above.

Preferably said kits contain an anti-C7orf9-, anti-C12orf7-, anti-MPP4 or anti-F379-antibody or a fragment thereof and/or a C7orf9-, C12orf7-, MPP4- or F379-specific nucleic acid probe.

Commonly used detection assays can comprise radioisotopic or non-radioisotopic methods. These comprise, inter alia, RIA (Radioisotopic Assay) and IRMA (Immune Radioimmunometric Assay), EIA (Enzyme Immuno Assay), ELISA (Enzyme-linked Immuno Assay), FIA (Fluorescent Immuno Assay), and CLIA (Chemoluminescent Immune Assay). Other detection methods that are used in the art are those that do not utilize tracer molecules. One prototype of these methods is the agglutination assay, based on the property of a given molecule to bridge at least two particles.

For diagnosis and quantification of (poly)peptides, polynucleotides, etc. in clinical and/or scientific specimens the immunological methods, as described above, are useful as well as molecular biological methods, like nucleic acid hybridization assays, PCR assays or DNA Enzyme Immunoassays (Mantero et al., Clinical Chemistry 37 (1991), 422-429) which are well known in the art. Further diagnostic methods leading to the detection of nucleic acid molecules in a sample comprise, e.g., ligase chain reaction (LCR), Southern blotting in combination with nucleic acid hybridization, comparative genome hybridization (CGH) or representative difference analysis (RDA). These methods are useful, e.g., for determining the expression of a nucleic acid molecule of the invention by detecting the presence of mRNA coding for a protein of the invention which comprises, for example, obtaining mRNA from cells of a subject and contacting the mRNA so obtained with a probe/primer comprising a nucleic acid molecule capable of specifically hybridizing with a nucleic acid molecule of the invention under suitable conditions (see also supra), and detecting the presence and/or determining the concentration of mRNA hybridized to the probe/primer. These methods are known in the art and can be carried out without any undue experimentation. The above approaches can also be used for the detection of mutations or chromosomal rearrangements.

The kit of the invention may comprise one or more containers filled with, for example, one or more probes (reagents) of the invention. Associated with container(s) of the kit can be a notice in the form prescribed by a governmental agency regulating the manufacture, use or sale of pharmaceuticals or biological products, which notice reflects approval by the agency of manufacture, use or sale for human administration.

The provision of the nucleic acid molecules according to the invention also opens up the possibility to produce transgenic non-human animals showing, e.g., a reduced level of the proteins as described above. Techniques how to achieve this are well known to the person skilled in the art. Thus, the present invention also relates to a method for the production of a transgenic non-human animal, preferably transgenic mouse, comprising introduction of a nucleic acid molecule or vector of the invention into a germ cell, an embryonic cell, stem cell or an egg or a cell derived therefrom. The non-human animal can be a non-transgenic healthy animal, or may have a disorder caused by at least one mutation in the C7orf9-, C12orf7-, MPP4- or F379-protein. Such transgenic animals are well suited for, e.g., pharmacological studies of drugs in connection with mutant forms of the above described C7orf9-, C12orf7-, MPP4- and F379-proteins. Production of transgenic embryos and screening of those can be performed, e.g., as described by A. L. Joyner Ed., Gene Targeting, A Practical Approach (1993), Oxford University Press. The DNA of the embryonal membranes of embryos can be analyzed using, e.g., Southern blots with an appropriate probe; see supra. The invention also relates to transgenic non-human animals such as transgenic mouse, rats, hamsters, dogs, monkeys, rabbits, pigs etc. comprising a nucleic acid molecule or vector of the invention or obtained by the method described above, preferably wherein said nucleic acid molecule or vector is stably integrated into the genome of said non-human animal, preferably such that the presence of said nucleic acid molecule or vector leads to the expression of the C7orf9-, C12orf7-, MPP4- and/or F379-protein of the invention. Said animal may have one or several copies of the same or different nucleic acid molecules encoding one or several forms of the C7orf9-, C12orf7-, MPP4- or F379-protein or mutant forms thereof. This animal has numerous utilities, including as a research model for studying diseases like AMD and therefore, presents a novel and valuable animal in the development of therapies, treatment, etc. for such diseases. Accordingly, in this instance, the mammal is preferably non-human, e.g., a laboratory animal such as a mouse or rat.

The transgenic non-human animal may also show, for example, a deficiency in the expression of C7orf9, C12orf7, MPP4 and/or F379 compared to wild type animals due to the stable or transient presence of a foreign DNA resulting in at least one of the following features:

(a) disruption of (an) endogenous gene(s) encoding C7orf9, C12orf7, MPP4 and/or F379;

(b) expression of at least on antisense RNA and/or ribozyme against a transcript comprising a nucleic acid molecule(s) of the invention;

(c) expression of a non-translatable mRNA of the nucleic acid molecule(s) of the invention;

(d) expression of an antibody of the invention; or

(e) incorporation of a functional or non-functional copy of the gene(s) encoding C7orf9, C12orf7, MPP4 and/or F379.

Preferably, the transgenic non-human animal of the invention comprises at least one inactivated version of the C7orf9, C12orf7, MPP4 or F379 encoding nucleic acid molecule; see supra. This embodiment allows for example the study of the effect of various mutant forms of C7orf9-, C12orf7, MPP4- or F379-proteins on the onset of the clinical symptoms of the disease. All the applications that have been herein before discussed with regard to a transgenic animal also apply to animals carrying two, three or more transgenes. It might be also desirable to inactivate C7orf9-, C12orf7, MPP4- or F379-protein expression or function at a certain stage of development and/or life of the transgenic animal. This can be achieved by using, for example, tissue specific, developmental and/or cell regulated and/or inducible promoters which drive the expression of, e.g., an antisense or ribozyme directed against the C7orf9-, C12orf7-, MPP4- or F379-protein encoding mRNA; see also supra. A suitable inducible system is for example tetracycline-regulated gene expression as described, e.g., by Gossen and Bujard (Proc. Natl. Acad. Sci. 89 USA (1992), 5547-5551) and Gossen et al. (Trends Biotech. 12 (1994), 58-62). Similar, the expression of the mutant C7orf9-, C12orf7-, MPP4- or F379-protein may be controlled by such regulatory elements.

### EXAMPLES

The following Examples are intended to illustrate, but not to limit the invention. While such Examples are typical of those that might be used, other methods known to those skilled in the art may alternatively be utilized.

### EXAMPLE 1

### MPP4

### (A) Isolation of MPP4 CDNA

The publically accessible UniGene dataset, release no. 113 (June, 2000), at the National Center for Biotechnology Information (NCBI) at the National Institutes of Health (NIH), Bethesda, Md. (http://www.ncbi.nlm.nih.gov/UniGene/) was searched for human EST clusters consisting of ESTs exclusively derived from retina cDNA libraries or for EST clusters with an enrichment of retina ESTs, defined by a portion of retina ESTs that is greater than 30% of the total. One of the 1241 entries meeting these criteria, Hs.60673, contained EST sequences from the 5′- and 3′-ends of two nearly identical cDNA clones isolated from the Soares retina N2b4HR cDNA library (ze39a04, ze32b03) (http://www.ncbi.nlm.nih.gov/Genbank/GenbankOverview.html.) Reverse transcription (RT)-PCR using oligonucleotides A128F (5′-CTC ACA TCC TTC TCA GCC-3′) and A128R (5′-GTG GAA TGT CAG GGA AAT C-3′), priming to sequences in the 5′ reads of the cDNA clones, amplified a 193 bp transcript in retinal RNA but not in various other adult human tissues tested.

Inspection of the sequence of genomic clone NH0309N08 (GenBank Acc. No. AC007279) harbouring EST sequences from Hs.60673 revealed significant alignments with further ESTs derived from retina cDNA clones (ze27h05, ze30f10, zf58a06, ys72e09). On the basis of this additional cDNA sequence information, oligonucleotide primers A128F3 (5′-TGA CTG CCT CCA GGA ATT-3′), A128aF (5′-TTA CGA AAT GAA TGG GCG-3′), A128aR (5′-AGG CTC TAG GTC CAT GAC-3′) and A128R3 (5′-ATG TGA AAT CTG CGA AAG G-3′) were designed and used to amplify retinal RNA in RT-PCR assays. The RT-PCR fragments were completely sequenced with walking primer technology on a ABI 310 automated sequencer (Perkin Elmer, Norwalk, USA) using the ABI PRISM Ready Reaction Sequencing Kit (Perkin Elmer, Norwalk, USA). Assembly of the overlapping 1375 bp A128F3/A128aR- and the 786 bp A128aF/R3-amplified cDNA fragments as well as 414 bp of 5′ end sequence and 42 bp of the 3′ end sequence of cDNA clone ze27h05 yielded a 2435 bp transcript with a conserved polyadenylation signal at nucleotide position 2416 bp. It should be noted that this full length transcript does not include the 5′ end EST sequences of cDNA clones ze39a04 and ze32b03 (Hs.60673) which most likely have been derived from incompletely spliced mRNA precursor molecules.

The full length 2435 bp cDNA contains an open reading frame (ORF) of 1980 bp with a first potential in frame translation initiation codon, ATG, starting 69 nucleotides downstream (see Seq. ID No. 1). Therefore, the protein predicted from the ORF consists of 637 amino acid residues, resulting in a calculated molecular mass of 72.8 kDa and an isoelectric point of 5.4.

### (B) Expression Analysis

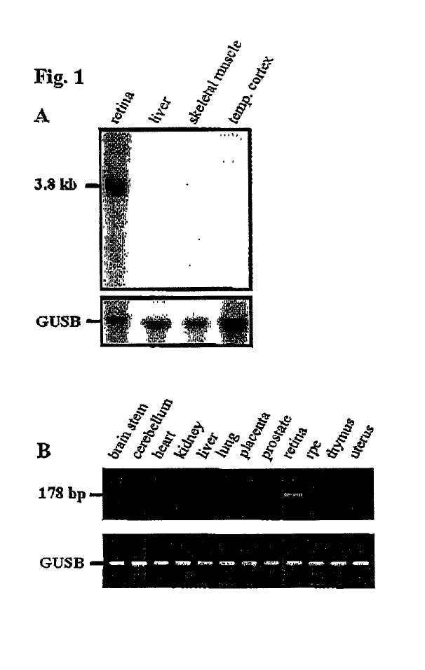

RT-PCR analysis using oligonucleotide primers A128F4 (5′-CGT GCC ATG ACT GAG TAC-3′) and A128aR (sequence described above) identified an 844 bp product in human retina. No PCR amplification was observed in cerebellum, brain stem, liver, lung, heart, thymus, placenta, uterus, prostate, retinal pigment epithelium (rpe) and kidney. Northern blot analysis was performed with total RNA isolated using the guanidinium thiocyanate method (Chomczynski and Sacchi, Anal.Biochem. 162 (1987), 156-159). Each lane containing 10 μg of total RNA from temporal cortex, muscle, retina and liver was electrophoretically separated in the presence of formaldehyde. A 327 bp DNA fragment from the 3′ untranslated region (UTR) was obtained by PCR amplification of genomic DNA with primer pair A128F6 (5′-AAC TGC AGT GGG TAC CAG-3′)/A126R6 (sequence described above) and was used as a probe for filter hybridization in 0.5 mM sodium phosphate buffer, pH 7.2; 7% SDS, 1 mM EDTA at 58° C. (Church and Gilbert, PNAS USA 81 (1984), 1991-1995). A single 3.8 kb transcript was identified exclusively in retina. The results of our expression analysis provide evidence that MPP4 is specific to the human retina. (FIG. 1).

### (C) Genomic Organization and Chromosomal Location of MPP4

To determine the exon/intron structure of MPP4, the 2435 bp cDNA sequence was aligned to the finished sequence of genomic clone NH0309N08 using the BLASTN program at NCBI (http://www.ncbi.nlm.nih.gov/cgi-bin/BLAST/nph-blast?Jform=1). This identified a total of 22 exons ranging from 15 bp to 493 bp. The putative translation start codon ATG is located in exon 2, the termination codon TGA in exon 22.

Genomic clone NH0309N08 contains DNA markers stSG2739 and sts-AA015777 which have been mapped to the D2S115-D2S307 interval on chromosome 2q31-2q33 by screening the Genebridge4 radiation hybrid panel (http://www.ncbi.nlm.nih.gov/genome/seq/ctg.cgi?tabview=M&BP=1000&CTG=Hs2_{—}2229&ORG=Hs).

### (D) Nucleotide and Protein Database Analyses

To find similar nucleotide sequences in the databases, the full length cDNA sequence of MPP4 was subjected to homology searches using the BLASTN program at NCBI. Significant sequence identity (85%) was found across with the entire 1325 bp of the annotated coding sequence as well as 250 bp of the 5′ UTR of the rat nRNA for rDLG6 (GenBank Acc. No. AB030499). The full length cDNA transcript of human MPP4 gene extends 253 bp in the 5′ direction in comparison with the known rDLG6 cDNA. Compared to the reported ORF in the rat this has extended the human MPP4 ORF and leads to an additional N-terminal 151 amino acids. Furthermore, the human transcript shows two insertions of 93 bp and 39 bp in the coding region corresponding to exon 12-15 and an elongated exon 17, resulting in the addition of further 44 amino acids. Immunological analyses indicated that rDLG6 is expressed predominantly in brain, however, expression studies in rat eye have not been performed.

Sequence alignment of the putative protein sequence of MPP4 with known proteins was done using the BLASTP and BEAUTY programs at Baylor College of Medicine (http://dot.imgen.bcm. tmc.edu:9331/seq-search/protein-search.html). The protein was also analzyed for specific motifs using the integration tool for the signature-recognition methods in InterPro at the European Bioinformatics Institute (http://www.ebi.ac.uk/interpro/interproscan/ipsearch.html). The 637 amino acids of the human MPP4 protein are 75% identical to the 441 amino acids of rat rDLG6 and similar to rDLG6, MPP4 shows the characteristic core structural organization of the MAGUK protein superfamily, with one PSD95/SAP90-Dlg-ZO-1 (PDZ) domain in the N-terminal half of the protein, a central src homology 3 (SH3) motif, and a C-terminal guanylate kinase-like (GUK) domain (Anderson, 1996 (Curr. Biol. 6 (1996) 382-384. Each of the different motifs is believed to be involved in protein-protein interactions (Anderson 1996). Furthermore, the GUK domain of the MAGUK protein CASK/LIN-2 has recently been demonstrated to regulate transcription in rat brain. Among the MAGUK proteins, human MPP4 is most similar to the p55-related MAGUK protein DLG3 of Danio rerio (39%, Acc. No. AAD39392), the discs large homolog 3 (Drosophila) of Mus musculus (37%, Acc. No. NP_{—}031889) and MPP3 (formerly termed as DLG3) of Homo sapiens (36%, Acc. No. NP_{—}001923). Local sequence comparisons showed 30-50% identity to the PDZ, SH3 and GUK domains of MAGUK family members.

The ubiquitious MAGUK proteins are localized at the plasma membrane of various animal cells where they are thought to contribute to signalling interactions as well as establishing and maintaining specialized structures of membranes. One of the flndamental roles of the MAGUK proteins is their ability to localise transmembrane proteins to specific sites, such as epithelial (e.g. ZO-1, ZO-2, ZO-3), septate junctions (e.g. Drosophila melanogaster dlg-1) and synapses (e.g. DLG1, PSD-95/SAP90/DLG4). For example, MPP1, a palmitoylated peripheral membrane phosphoprotein of human erythrocytes, links transmembrane proteins to the cortical actin cytoskeleton thereby modulating the shape of the cell.

Evidence for an important role in signalling pathways has initially been obtained by studies of MAGUK proteins in invertebrates. Lin-2 of Caenorhabditis elegans has been demonstrated to be involved in the signal propagation leading to vulval cell induction and certain mutations in Drosophila dlg-1 cause uncontrolled cell proliferation probably due to a defect in growth-inhibiting signals.

Most of the known functions of the MAGUK proteins are mediated through the 80-100 amino acids PDZ domains which bind to the extreme cytoplasmic carboxy-terminal tail of transmembrane proteins and other signal transduction proteins in a sequence and structure dependent manner. Recent investigations have shown that INAD, a protein with five PDZ domains, is an essential component of the visual transduction in Drosophila melanogaster. It organizes a minimum of seven proteins of the phototransduction cascade into a supramolecular signalling complex. This signalplex seems to promote the termination of the photoresponse and may also facilitate the rapid activation and amplification of the phototransduction cascade. PDZ-containing scaffold proteins may also coordinate signalling pathways of vertebrate phototransduction that simililarly require fast activation and deactivation as well as tight regulation. The importance of PDZ-containing proteins for retinal function has become evident by the more recent discovery of the PDZ domain-containing protein harnonin which is mutated in patients with Usher syndrome USH1C, a hereditary sensory disorder characterized by hearing loss and retinal degeneration.

### EXAMPLE 2

### C7orf9

### (A) Isolation of C7orf9 cDNA

The publically accessible UniGene dataset, release no. 113, was searched for human EST clusters consisting of ESTs exclusively derived from retina cDNA libraries or for EST clusters with an enrichment of retina ESTs, defined by a portion of retina ESTs that is greater than 30% of the total. One of the 1241 entries meeting these criteria, Hs.60473, contained approximately 350 bp of high quality EST sequences from the 3′-ends of two cDNA clones (ze34f06, ze37g05) isolated from the Soares retina N2b4HR cDNA library. The approximately 280 bp high quality EST sequences of the 5′-end of the cDNA clones available at the dbEST database (http://www2.ncbi.nlm.nih.gov/dbST/dbest_query.html) do not overlap with the corresponding 3′end ESTs.

To isolate further cDNA clones representing this gene, a retina lambda-Trip1Ex2 cDNA library was screened with a radio-labeled 199 bp DNA fragment obtained by PCR amplification of genomic DNA with primers A129F (5′-TCT GAG CCT AGA GGA TAC C-3′) and A129R (5′-GAT CTC AGA GGC AGG TTG-3′). Fourteen positive clones with inserts ranging from 0.5 to 1.6 kb were isolated and sequenced with walking primer technology on an ABI 310 automated sequencer (Perkin Elmer, Norwalk, USA) using the ABI PRISM Ready Reaction Sequencing Kit (Perkin Elmer, Norwalk, USA).

To isolate the complete 5′-end of the cDNA the technique of 5′-RACE (rapid amplification of cDNA ends) was used (Frohman et al. PNAS USA 85 (1988), 8998-9002). First strand cDNA synthesis was primed using the gene-specific antisense oligonucleotide A129R. Following cDNA synthesis, the first strand product was purified from unincorporated dNTPs and remaining primers A129R. A homopolymeric tail was then added to the 3′ end of the cDNA using terminal deoxynucleotidyl transferase (TdT) and dCTP. PCR amplification was accomplished using Taq DNA polymerase, the nested gene-specific primer A129R5 (5′-TGC TGT GAA GAT TGG AGA TC -3′) that anneals to a site located within the cDNA molecule, and a deoxyinosine-containing abridged anchor primer, AAP (5′-GGC CAC GCG TCG ACT AGT ACG GGI IGG GII GGG IIG-3′) provided by Life Technologies, Rockville, USA. To increase the quantity of the specific cDNA product the original PCR was re-amplified using the abridged universal amplification primer, AUAP (5′-GGC CAC GCG TCG ACT AGT AC-3′) provided by GIBCO Life Technologies, and a second nested gene-specific primer A129R4 (5′-AGC TTG AAG TGG CTA AAG TC-3′). Sequencing of the obtained PCR product using primer A129R4 did not reveal further upstream sequence suggesting that the identified cDNA sequence encompasses the complete 5′ sequences starting from the transcription start site of the transcript.

Assembly of the cDNA sequences yielded a 1190 bp cDNA sequence which contains an open reading frame (ORF) of 638 bp with a first potential in frame translation initiation codon, ATG, starting 47 nucleotides downstream (Seq. ID No. 26-28). The encoded putative protein consists of 196 amino acid residues and has a calculated molecular mass of 22.3 kDa and an isoelectric point of 9.26.

Comparison of 14 different cDNA sequences revealed the presence of a single nucleotide polymorphism (C/G) at position 143 bp causing the amino acid substitution isoleucine to methionine at codon 32 of the putative protein sequence.

### (B) Expression Analysis

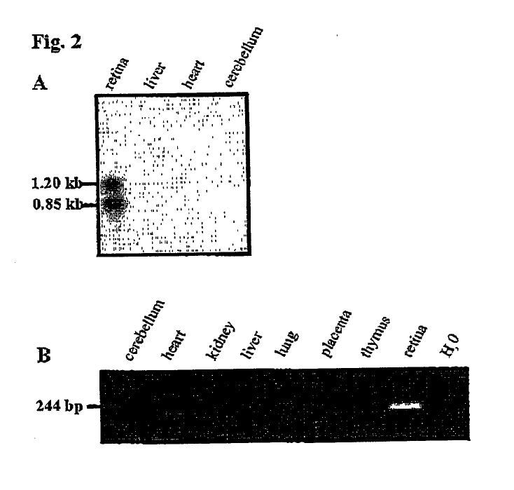

Reverse transcription-PCR analysis using oligonucleotide primer pairs A129F/A129R and A129F3 (5′-TGA TCT CCA ATC TTC ACA GC-3′)/A129R identified a specific 199 bp and 244 bp cDNA fragment in human retina only (FIG. 2). No PCR amplification was observed in human cerebellum, liver, lung, heart, placenta, thymus and kidney. Northern blot analysis was performed as described in Example 1. A 244 bp cDNA fragment from the 5′ region was used as a probe for filter hybridization in 0.5 mM sodium phosphate buffer, pH 7.2; 7% SDS, 1 mM EDTA at 58° C. Two transcripts of about 0.85 and 1.20 kb were identified exclusively in retina (FIG. 2).

### (C) Genomic Organization and Chromosomal Location of C7orf9

To determine the exon/intron structure of C7orf9, the 1190 bp cDNA sequence was aligned to the complete sequence of genomic BAC clone CTB-136N17 (GenBank Acc. No. AC004129) using the BLASTN program at NCBI. A total of 3 exons were identified with the putative translation start codon ATG located in exon 1 and the termination codon TAA in exon 3 (Seq. ID No. 26-28).

This genomic sequence of BAC clone CTB-136N17 contains DNA marker stSG51683 which has been mapped to the D7S2493-D7S529 interval on chromosome 7pl5-p21 by screening the Genebridge4 radiation hybrid panel (http://www.ncbi.nlm.nih.gov/genome/seq).

### (D) Nucleotide and Protein Database Analyses

The cDNA sequence of C7orf9 was subjected to homology searches using the BLASTN program at Baylor College of Medicine (BCM)and revealed 100% sequence identity between the coding region of C7orf9 and the human mRNA for RFamide-related peptide precursor (GenBank accession number AB040290). Therefore, the putative translation product of C7orf9 is identical to the RFamide-related peptide precursor (GenBank accession number BAB17674). The analysis for specific motifs using the integration tool for the signature-recognition methods in InterPro at the European Bioinformatics Institute. revealed that amino acids 99 to 109 and 138 to 148 demonstrate high similarity to the FARP (FMRFamide related peptide family) signature. RFamide-related peptides are generated by posttranslational processing of a precursor protein and are known to play a role in neurohormonal finctions, muscle contraction, and cardio-excitation.

### Example 3

### F379

### (A) Isolation of F379 EDNA

The publically accessible UniGene dataset, release no. 113 was searched for human EST clusters consisting of ESTs exclusively derived from retina cDNA libraries or for EST clusters with an enrichment of retina ESTs, defined by a portion of retina ESTs that is greater than 30% of the total. One of the 1241 entries meeting these criteria, Hs.35493, contained 22 EST sequences from the 5′-and/or 3′-ends of 15 cDNA clones isolated from the Soares retina N2b4HR cDNA library (ys82h08.rl, ys82h08.sl, ys66e12.rl, ys66e12.sl, ys84g04.rl, ze4g 02.rl, ys84c02.rl, ze42b07.sl, ze42b07.rl), the Nathans human retina cDNA randomly primed sublibrary (39a12) the Soares pineal gland N3HPG cDNA library (zf67e04.rl, zf67e04.sl, yt90d11rl, yt90d11.sl, yt84g01.rl, yt84g01.sl, yt83g01.sl, zf82e10.sl, zf82e10.rl, zf86d88.sl), the Soares fetal heart NbHH19W cDNA library (zd74d06.rl, zd74d06.sl) and the Soares testis NHT (ot33d09.sl) (http://www.ncbi.nlm.nih.gov/Genbank/GenbankOverview.html)

To identify the full length cDNA transcript of F379, human retinal libraries constructed in lambda-TripleEx2 and lambda-gt10 were screened. For each cDNA library, approximately 5×10⁵ plaques were probed with a alpha³²P-dCTP-labeled 328 bp fragment obtained by PCR amplification of retina cDNA using primer pair A071F (5′-TGT GCC AGG AAA GGA AGG-3′) and A071R (5′-TAG TCA GCA GCA TCG GGG G-3′). Three positive clones were isolated from the lambda-TripleEx2 retina cDNA library after second round screening and excised as plasmids from the phage vector following the instructions of the SMART™ library kit manual (Clontech, Palo Alto, USA). In the case of the lambda-gt10 cDNA library, one clone was isolated by PCR amplification. Primers A071F (described above) and lambda-gt10F (5′-AGC AAG TTC AGC CTG GTT AAG-3′) were used to amplify the clone from a mixed phage lysate containing the positive clone. Additionally, 750 bp of F379 cDNA was amplified from retina cDNA using primer pair A071F (described above) and A071R2 (5′-ATG TTC AGT CAG GCA GGG -3′). All cDNA library clones and PCR products were sequenced using the ABI PRISM Ready Reaction Sequencing Kit on an ABI 310 automated sequencer (Perkin Elmer, Norwalk, USA).

The 1188 bp full length consensus cDNA sequence of F379 (Seq.ID No.7) was determined from a compilation of the DNA sequences from the cDNA library clones, the PCR products and the ESTs of Hs.35493. An alignment of these sequences to the consensus cDNA sequence of F379 revealed that there were single base pair variations. These single base pair changes are summarized in Table 1. The full length consensus cDNA contained a putative open reading frame (ORF) of 85 amino acids (Seq. ID No. 31), starting at 347 bases from the most 5′ end of the full length consensus cDNA. The single base changes in the cDNA do not truncate the putative ORF by introducing a stop codon; rather, the variations cause amino acid substitutions or have no effect on the putative ORF (Table 1). The ORF contains Alu and MIR repetitive elements, which together account for 68 amino acids. The predicted protein has a calculated molecular mass of 9.2 KDa and an isoelectric point of 6.81.

TABLE 1
Single base variations in the cDNA sequence
and their associated amino acid changes

| Position from | | |
|---|---|---|
| beginning of | Nucleotide | Amino Acid |
| cDNA | Change | Change |
| 325 | G | n/a* |
| 429 | T | L |
| 442 | A | R |
| 528 | T | I |
| 557 | T | S |
| 932 | A | n/a* |
| 971 | C | n/a* |
| 987 | T | n/a* |

*single base pair variation is located outside of putative ORF

### (B) Expression Analysis

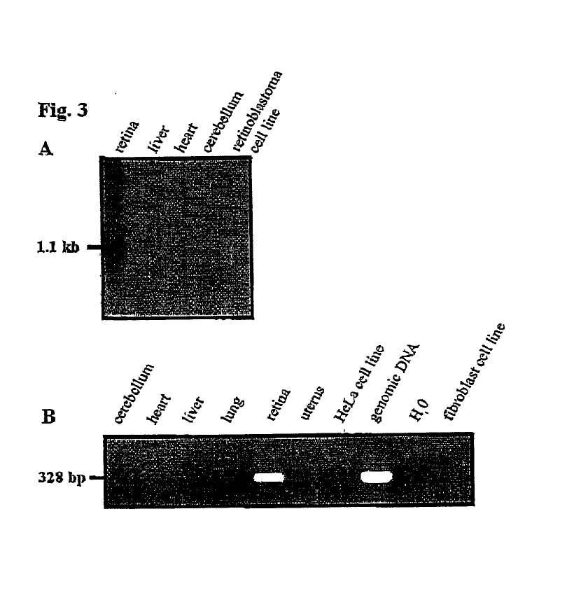

Reverse transcription-polymerase chain reaction (RT-PCR) using oligonucleotides A071F and A071R, priming to sequences in the 5′ reads of the cDNA clones, amplified a 328 bp transcript from human retina RNA but not from uterus, cerebellum, heart, liver or lung RNA. Furthermore, Northern blot analysis was performed as described in Example 1. A 219 bp DNA fragment from the 3′ region of the gene was obtained by PCR amplification of genomic DNA with primer pair A071F3 (5′-TTC TTG TCG GAT GCC CTC-3′) and A071R2 (described above). This DNA fragment was used as a probe for filter hybridization in 0.5 mM sodium phosphate buffer, pH 7.2; 7% SDS, 1 mM EDTA at 58° C. A single transcript of about 1.1 kb was identified only in retina The results of the expression analysis show that F379 is found exclusively in retina (FIG. 3). Furthermore, the size of the transcript detected by Northern blot correlates to the size of the full length cDNA consensus sequence (1188 bp).

### (C) Genomic Organization and Chromosomal Location of F379

To determine the exon/intron structure of F379, the 1188 bp consensus cDNA sequence was aligned to the finished and unfinished genomic sequences using the BLASTN program at NCBI. The complete cDNA sequence of F379 aligned to genomic clones from different chromosomes, including chromosome 19 (LLNLR-222A1), chromosome 22 (RP11-395L14), chromosome 2 (RP11-559H14), chromosome 21 (RP11-34P13), chromosome 10 (RP11-438F6), chromosome 12 (RP11-598F7), and chromosome 9 (RP11-142M1). Partial alignments were also found to genomic clones from chromosome 15 (15qtel_c184at3), chromosome 12 (12PTEL057, 12PTEL055, RPCI11-55L14) and chromosome 19 (CTD-2102P23). These alignments identified three exons ranging from 205 bp to 621 bp. The putative translation start codon ATG is located in exon 1 and the termination codon TGA is located in exon 3.

PCR-based screening of two different human/rodent somatic cell hybrid DNA mapping panels also indicated the multicopy nature of F379. A commercial human/rodent somatic cell hybrid mapping panel (Mapping Panel 2 from Coriell Institute for Medical Research, Camden, USA) was screened with primer set A071F (described above) and A071R (described above), yielding a 328 bp product in cell line DNA containing chromosomes 2, 3, 6, 9, 12, 15, 19, and 20. Based on this result, gene names D2F379S1E, D3F379S2E, D6F379S3E, D9F379S4E, D12F379S5E, D15F379S6E, D19F379S7E, and D20F379S assigned to chromosomes 2, 3, 6, 9, 12, 15, 19, and 20, respectively by the Genome Database (http://www.gdb.org/). The multi-chromosomal location of F379 is consistent with that of cosmid clone F7501, which is overlapping with two completely sequenced BAC clones (RP11-395L14 and LLNLR-222A1, see above). This cosmid has been shown to be a part of a sub-telomeric block which is present at 1q, 2q13-14, 3q, 5q, 6p, 6q, 8p, 9p, 9q, 11p, 12p, 15q, 19p, 20p, and 20q, as shown by fluorescence in-situ hybridization (FISH) analysis (Trask et al., Hum.Mol.Genet. 9 (1998), 1329-1349).

### (D) Nucleotide and Protein Database Analyses

Sequence alignments of the complete consensus cDNA sequence were done using the BLASTN program at NCBI. Other than the EST and genomic sequences described above and the matches to Alu or MIR repeat elements, no significant matches to characterized genes were found.

Comparison of the putative ORF to known proteins was done using the BLASTP program at NCBI. Sequence alignments to other proteins were localized to the region of the amino acids coded by the Alu repeat. No other significant matches were found. The protein was also analyzed for specific motifs using the integration tool for the signature-recognition methods in InterPro at the European Bioinformatics Institute (http://www.enzim.hu/hmmtop/) No motifs or patterns were found. The ORF has no predicted transmembrane regions as analysed by HMMTOP program (http://www.enzim.hu/hmmtop/) and the TMHMM program (http://www.cbs.dtu.dk/services/TMHMM-1.0/). There are two potential GalNAc O-glycosylation sites at amino acids 23 and 27, as determined by the NetOGlyc 2.0 Prediction Server (http://www.cbs.dtu.dk/services/NetOGlyc/). A N-glycosylation site was predicted at amino acid 51 using the PROSITE SCAN program at 90% similiarity (http://pbil.ibcp.fr/cgi-bin/npsa_automat.pl?page=npsa_prosite.html).

### Example 4

### C12orf7

### (A) Isolation of C12orf7 CDNA

The publicly accessible UniGene dataset, release no. 113, was searched for human EST clusters consisting of ESTs exclusively derived from retina cDNA libraries or for EST clusters with an enrichment of retina ESTs, defined by a portion of retina ESTs that is greater than 30% of the total. One of the 1241 entries meeting these criteria, Hs.2841 1, contained 10 EST sequences. Eight ESTs represent the 5′- and 3′-ends of four cDNA clones isolated from the Soares retina N2b4HR cDNA library (zf50g06, ze44g08, yt72c07, zf52h05) and two represent the 3′-ends of two cDNA clones isolated from the Soares placenta Nb2HP cDNA library (yi08f03.sl, yi75a07.sl).

To identify the full length cDNA transcript of C12orf7, a lambda-gt10 retina cDNA library was probed with a alpha³²P-dCTP-labeled 863 bp fragment obtained by PCR amplification of cDNA clone zf50g06 using primer pair A038F3 (5′-CGG AAC CGC TGT GAG TGC-3′) and A038F (5′-TAG GCA GAG GTG GAT GGG-3′). The inserts of eleven positive clones were sequenced with walking primer technology using the ABI PRISM Ready Reaction Sequencing Kit on an ABI 310 automated sequencer (Perkin Elmer, Norwalk, USA).

Compilation of the 11 cDNA sequences revealed two different cDNA species. One cDNA molecule consists of 1428 bp, the second cDNA sequence contains an insertion of 30 bp at nucleotide position 549. To isolate the complete 5′-end of the cDNA the technique of 5′-RACE (rapid amplification of cDNA ends) was used as described in Example 2 except that first strand cDNA synthesis was primed with the gene-specific antisense oligonucleotide A038F and PCR amplification was accomplished using the gene-specific primer A038R3 (5′-GGC CAC TCG GGC TTG TAG-3′) and a second nested gene-specific primer A038R4 (5′-GTG CAA TGC CAG CTC TTC-3′). Sequencing of the obtained PCR product using primer A038R4 revealed an additional 86 bp of 5′ sequence. Assembly of the 5′-RACE sequence and the cDNA sequences obtained from the cDNA clones yielded a 1514 (Seq. ID No. 35) and a 1544 bp transcript (Seq. ID No. 36).

Comparison of the cDNA sequences revealed the presence of two single nucleotide polymorphisms at position 40 bp (A/T) and 88 bp (C/T) of Seq. ID No. 35 and 36.

Both cDNA variants contain the same putative open reading frame (ORF) encoding a 345 amino acid (aa) (Seq. ID No. 37) and a 355 aa (Seq. ID No. 38) protein. The putative proteins share the same potential in frame initiation codon, ATG, located 154 nucleotides downstream of the most 5′ cDNA sequence. The putative protein sequences No. 11a and No. 11b have a calculated molecular mass of 37.1 kD and 38.0 kD and an isoelectric point of 5.59 and 5.49, respectively.

### (B) Expression Analysis

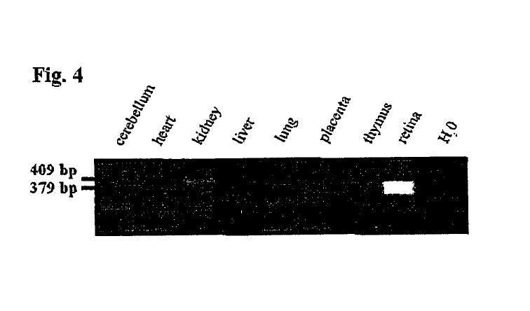

Reverse transcription-PCR using oligonucleotides A038F and A038R (5′-TGC CAA GCT GTT AGT GCC-3′), priming to the 3′ end of the cDNA sequence, amplified a 231 bp cDNA fragment from human retina RNA but not from human brain, heart, liver, lung or uterus RNA. RT-PCR using primers A038F4 (5′-CAT GCT ACC ACG GCT TCC-3′) and A038R3 amplified a 379 bp and 409 bp fragment from human retina RNA but not from human cerebellum, heart, kidney, liver, lung, placenta or thymus RNA (example in FIG. 4).

### (C) Genomic Organization and Chromosomal Location of C12orf7

To determine the exon/intron structure of C12orf7, the cDNA sequences were aligned to the unfinished genomic sequence of clone RP11-1100L3 (GenBank accession number AC025259) using the BLASTN program at NCBI. Six exons ranging from 143 bp to 477 bp were identified (Seq. ID No. 39-45). The putative translation start codon ATG is located in exon 2 and the termination codon TAA is located in exon 6. The insertion in cDNA sequence No. lOb was identified as a 30 bp extension of exon 4 generated by the use of an alternative splice donor consensus sequence. Both splice donor sites have similar splicing scores.

Radiation hybrid mapping using the Genebridge4 panel has localized Hs.28411 between the markers D12S333-D12S325 on chromosome 12q11.1-13.2 (http://www.ncbi.nhn.nih.gov/genome/sts/sts.cgi?uid=92710). In addition, genomic clone RP11-1100L3 has been mapped to chromosome 12 (Genbank accession number. AC025259).

### (D) Nucleotide and Protein Database Analyses

Sequence alignments of the C12orf7 cDNA sequences to known nucleotide sequences were done using the BLASTN program at BCM. No significant matches to known gene sequences were identified. A LINE/L1 repeat was found in the 3′ untranslated region at position 1281-1403 bp (Seq. ID No. 35) and 1311-1433 bp (Seq. ID No. 36).

Comparison of the putative translation products of C7orf9 against protein databases was performed using the BLASTP and BEAUTY programs at BCM (http://dot.imgen.bcm.tmc.edu:9331/seq-search/protein-search.html). The proteins were also analzyed for motifs and patterns using the integration tool for the signature-recognition methods in InterPro at the European Bioinformatics Institute (http://www.ebi.ac.uk/interpro/interproscan/ipsearch.html). Two ankyrin repeats at position 112-144 aa and 147-179 aa were identified in the longer protein isoform (Seq. ID No. 38), whereas only one ankyrin repeat at position 112-144 aa was identified in the shorter protein isoform (Seq. ID No. 37). The approximately 33 residue ankyrin domain is found in many finctionally unrelated proteins and is known to play a role in protein-protein interactions. No significant homology was found to known protein sequences. No transmembrane regions were predicted by the HMMTOP (http://www.enzim.hu/hmmtop/) or TMHMM program (http://www.cbs.dtu.dk/services/TMHMM-1.0/).

The foregoing is meant to illustrate, but not to limit, the scope of the invention. The person skilled in the art can readily envision and produce further embodiments, based on the above teachings, without undue experimentation.

Priority application U.S. application Ser. No. 60/253,751, filed Nov. 29, 2000, including the specification, drawings, claims, and abstract, is hereby incorporated by reference. All publications cited herein are incorporated in their entireties by reference.

1. An isolated nucleic acid molecule encoding the retina-specific human protein C7orf9, C12orf7, MPP4 or F379 or a protein exhibiting biological properties of C7orf9, C12orf7, MPP4 or F379 being selected from the group consisting of
(a) a nucleic acid molecule encoding a protein that comprises the amino acid sequence depicted in Seq. ID No. 24, 29, 31, 37 or 38;
(b) a nucleic acid molecule comprising the nucleotide sequence depicted in Seq. Id no. 2-23, 26-28, 32-34, 35 or 36;
(c) a nucleic acid molecule comprising the nucleotide sequence depicted in Seq. ID No. 1, 25, 30 or 39-45;
(d) a nucleic acid molecule which hybridizes to a nucleic acid molecule specified in (a) to (c);
(e) a nucleic acid molecule the nucleic acid sequence of which deviates from the nucleic sequences specified in (a) to (d) due to the degeneration of the genetic code; and
(f) a nucleic acid molecule, which represents a fragment, derivative or allelic variation of a nucleic acid sequence specified in (a) to (e).

2. A recombinant vector containing a nucleic acid molecule of claim 1.

3. The recombinant vector of claim 2 wherein the nucleic acid molecule is operatively linked to regulatory elements allowing transcription and synthesis of a translatable RNA in prokaryotic and/or eukaryotic host cells.

4. A recombinant host cell which contains the recombinant vector of claim 3.

5. The recombinant host cell of claim 4, which is a mammalian cell, a bacterial cell, an insect cell or a yeast cell.

6. An isolated protein exhibiting biological properties of the retina-specific human protein C7orf9, C12orf7, MPP4 or F379 which is encoded by a nucleic acid molecule of claim 1.

7. A recombinant host cell that expresses the isolated protein of claim 6.

8. A method of making an isolated protein exhibiting biological properties of the retina-specific human protein C7orf9, C12orf7, MPP4 or F379 comprising:
(a) culturing the recombinant host cell of claim 6 under conditions such that said protein is expressed; and
(b) recovering said protein.

9. The protein produced by the method of claim 8.

10. A nucleic acid molecule of at least 15 nucleotides in length hybridizing specifically with a nucleic acid molecule of claim 1 or with a complementary strand thereof.

11. The nucleic acid molecule of claim 10, which is an antisense RNA characterized in that it is complementary to an mRNA transcribed from a nucleic acid molecule of claim 1 or a part thereof and can selectively bind to said mRNA or part thereof, said sequence being capable of inhibiting the synthesis of the protein encoded by said nucleic acid molecule.

12. The nucleic acid molecule of claim 10 which is a ribozyme characterized in that it is complementary to an mRNA transcribed from a nucleic acid molecule of claim 1 or a part thereof and can selectively bind to and cleave said mRNA or part thereof, thus inhibiting the synthesis of the protein encoded by said nucleic acid molecule.

13. An inhibitor characterized in that it can suppress the activity of a protein of claim 6.

14. A method for diagnosing macular degeneration or a predisposition for macular degeneration which comprises contacting a target sample suspected to contain the retina-specific human protein C7orf9, C12orf7, MPP4 and/or F379 or the C7orf9, C12orf7, MPP4 and/or F379 encoding nucleic acid with a reagent which reacts with the C7orf9, C12orf7, MPP4 and/or F379 protein and/or C7orf9, C12orf7, MPP4 and/or F379 encoding nucleic acid and detecting the C7orf9, C12orf7, MPP4 and/or F379 protein and/or C7orf9, C12orf7, MPP4 and/or F379 encoding nucleic acid, wherein the presence of a mutation within the C7orf9, C12orf7, MPP4 and/or F379 encoding nucleic acid, a chromosal rearrangement or abnormal levels of the C7orf9, C12orf7, MPP4 and/or F379 protein and/or C7orf9, C12orf7, MPP4 and/or F379 encoding mRNA are indicative for macular degeneration or a predisposition for macular degeneration.

15. The method of claim 14, wherein the macular degeneration is AMD.

16. The method of claim 14, wherein the reagent is a C7orf9-, C12orf7-, MPP4- or F379-specific nucleic acid probe.

17. The method of claim 14, wherein the reagent is an anti-C7orf9-, anti-C12orf7-, anti-MPP4 or anti-F379-antibody.

18. The method of claim 14, wherein the reagent is detectably labeled.

19. The method of claim 18, wherein the label is selected from the group consisting of a radioisotope, a bioluminescent compound, a chemoluminescent compound, a fluorescent compound, a metal chelate, or an enzyme.

20. A method for treating macular degeneration or a predisposition for macular degeneration which comprises administering to a mammalian subject a therapeutically effective amount of a reagent which decreases, inhibits or increases expression of C7orf9, C12orf7, MPP4 and/or F379 or which leads to the expression of a biologically active C7orf9, C12orf7, MPP4 and/or F379 protein.

21. The method of claim 20, wherein the macular degeneration is AMD.

22. The method of claim 20, wherein the reagent is a nucleotide sequence comprising an antisense RNA characterized in that it is complementary to an mRNA transcribed from a nucleic acid molecule of claim 1 or a part thereof and can selectively bind to said mRNA or part thereof, said sequence being capable of inhibiting the synthesis of the protein encoded by said nucleic acid molecule.

23. The method of claim 20, wherein the reagent is a nucleotide sequence comprising a ribozyme characterized in that it is complementary to an mRNA transcribed from a nucleic acid molecule of claim 1 or a part thereof and can selectively bind to and cleave said mRNA or part thereof, thus inhibiting the synthesis of the protein encoded by said nucleic acid molecule.

24. The method of claim 20, wherein the reagent is an inhibitor of C7orf9-, C12orf7-, MPP4- and/or F379-protein.

25. The method of claim 24, wherein the inhibitor is an anti-C7orf9-, anti-C12orf7-, anti-MPP4- or anti-F379-antibody or a fragment thereof.

26. The method of claim 20, wherein the reagent is the recombinant vector of claim 2.

27. The method of claim 20, wherein the reagent is an isolated protein of claim 6.

28. A diagnostic kit useful for the detection of macular degeneration or a predisposition for macular degeneration containing an anti-C7orf9-, anti-C12orf7-, anti-MPP4 or anti-F379-antibody or a fragment thereof and/or a C7orf9-, C12orf7-, MPP4- or F379-specific nucleic acid probe.

29. A transgenic non-human animal comprising at least one nucleic acid molecule of claim 1.

30. A transgenic non-human animal comprising at least one inactivated version of the C7orf9, C12orf7, MPP4 or F379 encoding nucleic acid molecule.

31. The transgenic non-human animal of claim 30 which is a mouse or a rat.
